(19) United States
(12) Patent Application Publication
(10) Pub. No.: US 2018/0353483 A1
(43) Pub. Date: Dec. 13, 2018

(54) PHARMACEUTICAL COMPOSITIONS AND METHODS FOR INDOLEAMINE, 2, 3-DIOXYGENASE INHIBITION AND INDICATIONS THEREFOR

(71) Applicant: Incyte Corporation, Wilmington, DE (US)

(72) Inventors: Krishnaswamy Yeleswaram, Landenberg, PA (US); Jack Guoen Shi, Wilmington, DE (US)

(21) Appl. No.: 15/773,454

(22) PCT Filed: Nov. 4, 2016
(86) PCT No.: PCT/US2016/060693

§ 371 Date: May 3, 2018

Related U.S. Application Data
(60) Provisional application No. 62/250,968, filed on Nov. 4, 2015.

(51) Int. Cl. A61K 31/4245; A61K 9/20; A61P 35/02

(52) U.S. Cl. CPC A61K 31/4245; A61K 9/2054; A61K 9/2027; A61K 45/06; A61K 9/2009; A61P 35/02; A61K 9/2018

(57) ABSTRACT

The present invention is directed to pharmaceutical compositions of an inhibitor of indoleamine 2,3-dioxygenase and are useful in the treatment of cancer and other disorders.

### CROSS-REFERENCE TO RELATED APPLICATIONS

This application claims priority to U.S. Provisional Patent Application No. 62/250,968, filed on Nov. 4, 2016, which is hereby incorporated by reference in its entirety.

### FIELD OF INVENTION

The present invention is directed to pharmaceutical compositions of an inhibitor of indoleamine 2,3-dioxygenase and are useful in the treatment of cancer and other disorders.

### BACKGROUND OF THE INVENTION

Tryptophan (Trp) is an essential amino acid required for the biosynthesis of proteins, niacin and the neurotransmitter 5-hydroxytryptamine (serotonin). The enzyme indoleamine 2,3-dioxygenase (also known as INDO, IDO or IDO1) catalyzes the first and rate limiting step in the degradation of L-tryptophan to N-formyl-kynurenine. In human cells, a depletion of Trp resulting from IDO activity is a prominent gamma interferon (IFN-γ)-inducible antimicrobial effector mechanism. IFN-γ stimulation induces activation of IDO, which leads to a depletion of Trp, thereby arresting the growth of Trp-dependent intracellular pathogens such as Toxoplasma gondii and Chlamydia trachomatis. IDO activity also has an antiproliferative effect on many tumor cells, and IDO induction has been observed in vivo during rejection of allogeneic tumors, indicating a possible role for this enzyme in the tumor rejection process (Daubener, et al., 1999, Adv. Exp. Med. Biol., 467: 517-24; Taylor, et al., 1991, FASEB J., 5: 2516-22).

It has been observed that HeLa cells co-cultured with peripheral blood lymphocytes (PBLs) acquire an immuno-inhibitory phenotype through up-regulation of IDO activity. A reduction in PBL proliferation upon treatment with interleukin-2 (IL2) was believed to result from IDO released by the tumor cells in response to IFNG secretion by the PBLs. This effect was reversed by treatment with 1-methyl-tryptophan (1MT), a specific IDO inhibitor. It was proposed that IDO activity in tumor cells may serve to impair antitumor responses (Logan, et al., 2002, Immunology, 105: 478-87).

Recently, an immunoregulatory role of Trp depletion has received much attention. Several lines of evidence suggest that IDO is involved in induction of immune tolerance. Studies of mammalian pregnancy, tumor resistance, chronic infections and autoimmune diseases have shown that cells expressing IDO can suppress T-cell responses and promote tolerance. Accelerated Trp catabolism has been observed in diseases and disorders associated with cellular immune activation, such as infection, malignancy, autoimmune diseases and AIDS, as well as during pregnancy. For example, increased levels of IFNs and elevated levels of urinary Trp metabolites have been observed in autoimmune diseases; it has been postulated that systemic or local depletion of Trp occurring in autoimmune diseases may relate to the degeneration and wasting symptoms of these diseases. In support of this hypothesis, high levels of IDO were observed in cells isolated from the synovia of arthritic joints. IFNs are also elevated in human immunodeficiency virus (HIV) patients and increasing IFN levels are associated with a worsening prognosis. Thus, it was proposed that IDO is induced chronically by HIV infection, and is further increased by opportunistic infections, and that the chronic loss of Trp initiates mechanisms responsible for cachexia, dementia and diarrhea and possibly immunosuppression of AIDS patients (Brown, et al., 1991, Adv. Exp. Med. Biol., 294: 425-35). To this end, it has recently been shown that IDO inhibition can enhance the levels of virus-specific T cells and, concomitantly, reduce the number of virally-infected macrophages in a mouse model of HIV (Portula et al., 2005, Blood, 106: 2382-90).

IDO is believed to play a role in the immunosuppressive processes that prevent fetal rejection in utero. More than 40 years ago, it was observed that, during pregnancy, the genetically disparate mammalian conceptus survives in spite of what would be predicted by tissue transplantation immunology (Medawar, 1953, Symp. Soc. Exp. Biol. 7: 320-38). Anatomic separation of mother and fetus and antigenic immaturity of the fetus cannot fully explain fetal allograft survival. Recent attention has focused on immunologic tolerance of the mother. Because IDO is expressed by human syncytiotrophoblast cells and systemic tryptophan concentration falls during normal pregnancy, it was hypothesized that IDO expression at the maternal-fetal interface is necessary to prevent immunologic rejection of the fetal allografts. To test this hypothesis, pregnant mice (carrying syngeneic or allogeneic fetuses) were exposed to 1MT, and a rapid, T cell-induced rejection of all allogeneic concept was observed. Thus, by catabolizing tryptophan, the mammalian conceptus appears to suppresses T-cell activity and defends itself against rejection, and blocking tryptophan catabolism during murine pregnancy allows maternal T cells to provoke fetal allograft rejection (Munn, et al., 1998, Science, 281: 1191-3).

Further evidence for a tumoral immune resistance mechanism based on tryptophan degradation by IDO comes from the observation that most human tumors constitutively express IDO, and that expression of IDO by immunogenic mouse tumor cells prevents their rejection by preimmunized mice. This effect is accompanied by a lack of accumulation of specific T cells at the tumor site and can be partly reverted by systemic treatment of mice with an inhibitor of IDO, in the absence of noticeable toxicity. Thus, it was suggested that the efficacy of therapeutic vaccination of cancer patients might be improved by concomitant administration of an IDO inhibitor (Uyttenhove et al., 2003, Nature Med., 9: 1269-74). It has also been shown that the IDO inhibitor, 1-MT, can synergize with chemotherapeutic agents to reduce tumor growth in mice, suggesting that IDO inhibition may also enhance the anti-tumor activity of conventional cytotoxic therapies (Muller et al., 2005, Nature Med., 11: 312-9).

One mechanism contributing to immunologic unresponsiveness toward tumors may be presentation of tumor antigens by tolerogenic host APCs. A subset of human IDO-expressing antigen-presenting cells (APCs) that coexpressed CD123 (IL3RA) and CCR6 and inhibited T-cell proliferation have also been described. Both mature and immature CD123-positive dendritic cells suppressed T-cell activity, and this IDO suppressive activity was blocked by 1MT (Munn, et al., 2002, Science, 297: 1867-70). It has also been demonstrated that mouse tumor-draining lymph nodes (TDLNs) contain a subset of plasmacytoid dendritic cells (pDCs) that constitutively express immunosuppressive levels of IDO. Despite comprising only 0.5% of lymph node cells, in vitro, these pDCs potently suppressed T cell responses to antigens presented by the pDCs themselves and also, in a dominant fashion, suppressed T cell responses to third-party antigens presented by nonsuppressive APCs. Within the population of pDCs, the majority of the functional IDO-mediated suppressor activity segregated with a novel subset of pDCs coexpressing the B-lineage marker CD19. Thus, it was hypothesized that IDO-mediated suppression by pDCs in TDLNs creates a local microenvironment that is potently suppressive of host antitumor T cell responses (Munn, et al., 2004, J. Clin. Invest., 114(2): 280-90).

IDO degrades the indole moiety of tryptophan, serotonin and melatonin, and initiates the production of neuroactive and immunoregulatory metabolites, collectively known as kynurenines. By locally depleting tryptophan and increasing proapoptotic kynurenines, IDO expressed by dendritic cells (DCs) can greatly affect T-cell proliferation and survival. IDO induction in DCs could be a common mechanism of deletional tolerance driven by regulatory T cells. Because such tolerogenic responses can be expected to operate in a variety of physiopathological conditions, tryptophan metabolism and kynurenine production might represent a crucial interface between the immune and nervous systems (Grohmann, et al., 2003, Trends Immunol., 24: 242-8). In states of persistent immune activation, availability of free serum Trp is diminished and, as a consequence of reduced serotonin production, serotonergic functions may also be affected (Wirleitner, et al., 2003, Curr. Med. Chem., 10: 1581-91).

Interestingly, administration of interferon-α has been observed to induce neuropsychiatric side effects, such as depressive symptoms and changes in cognitive function. Direct influence on serotonergic neurotransmission may contribute to these side effects. In addition, because IDO activation leads to reduced levels of tryptophan, the precursor of serotonin (5-HT), IDO may play a role in these neuropsychiatric side effects by reducing central 5-HT synthesis. Furthermore, kynurenine metabolites such as 3-hydroxy-kynurenine (3-OH-KYN) and quinolinic acid (QUIN) have toxic effects on brain function. 3-OH-KYN is able to produce oxidative stress by increasing the production of reactive oxygen species (ROS), and QUIN may produce overstimulation of hippocampal N-methyl-D-aspartate (NMDA) receptors, which leads to apoptosis and hippocampal atrophy. Both ROS overproduction and hippocampal atrophy caused by NMDA overstimulation have been associated with depression (Wichers and Maes, 2004, J. Psychiatry Neurosci., 29: 11-17). Thus, IDO activity may play a role in depression.

In light of the experimental data indicating a role for IDO in immunosuppression, tumor resistance and/or rejection, chronic infections, HIV-infection, AIDS (including its manifestations such as cachexia, dementia and diarrhea), autoimmune diseases or disorders (such as rheumatoid arthritis), and immunologic tolerance and prevention of fetal rejection in utero, therapeutic agents aimed at suppression of tryptophan degradation by inhibiting IDO activity are desirable. Inhibitors of IDO can be used to activate T cells and therefore enhance T cell activation when the T cells are suppressed by pregnancy, malignancy or a virus such as HIV. Inhibition of IDO may also be an important treatment strategy for patients with neurological or neuropsychiatric diseases or disorders such as depression.

Small molecule inhibitors of IDO are being developed to treat or prevent IDO-related diseases such as those described above. For example, oxadiazole and other heterocyclic IDO inhibitors are reported in US 2006/0258719 and US 2007/0185165. PCT Publication WO 99/29310 reports methods for altering T cell-mediated immunity comprising altering local extracellular concentrations of tryptophan and tryptophan metabolites, using an inhibitor of IDO such as 1-methyl-DL-tryptophan, p-(3-benzofuranyl)-DL-alanine, p-[3-benzo(b)thienyl]-DL-alanine, and 6-nitro-L-tryptophan) (Munn, 1999). Reported in WO 03/087347, also published as European Patent 1501918, are methods of making antigen-presenting cells for enhancing or reducing T cell tolerance (Munn, 2003). Compounds having indoleamine-2,3-dioxygenase (IDO) inhibitory activity are further reported in WO 2004/094409; and U.S. Patent Application Publication No. 2004/0234623 is directed to methods of treating a subject with cancer or an infection by the administration of an inhibitor of indoleamine-2,3-dioxygenase in combination with other therapeutic modalities. An example of IDO inhibitor is 4-({2-[(aminosulfonyl)amino]ethyl}amino)-N-(3-bromo-4-fluorophenyl)-N′-hydroxy-1,2,5-oxadiazole-3-carboximidamide, which is described in U.S. Pat. No. 8,088,803. There remains a need for new pharmaceutical compositions having suitable properties useful in the treatment of IDO-related diseases. The present invention described herein is directed toward this end.

### SUMMARY OF THE INVENTION

The present invention provides, inter alia, a method of treating cancer in a patient comprising administering to said patient a pharmaceutical composition comprising Compound 1,

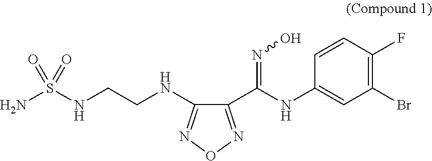 [SMILES: NS(=O)(=O)NCCNc1nonc1C(=NO)Nc1ccc(F)c(Br)c1]

or a pharmaceutically acceptable salt thereof, and one or more excipients, wherein the treating comprises a dosage regimen comprising from about 25 mg to about 700 mg on a free basis of Compound 1, or a pharmaceutically acceptable salt thereof, administered orally twice daily.

The present invention also provides a method of treating cancer in a patient comprising administering to said patient a pharmaceutical composition comprising Compound 1, or a pharmaceutically acceptable salt thereof, and one or more excipients, wherein the treating comprises a dosage regimen which attains at steady state, an Iₘᵢₙ of about 50% or greater, or an I_{avg} of about 70% or greater.

The present invention also provides a method of treating cancer in a patient comprising administering to said patient a pharmaceutical composition comprising Compound 1, or a pharmaceutically acceptable salt thereof, and one or more excipients, wherein the treating comprises a dosage regimen which attains at steady state:

(1) a Cₘₐₓ from about 0.10 μM to about 10 μM, a Cₘᵢₙ from about 0.01 μM to about 2.0 μM, a Tₘₐₓ of about 1 h to about 6 h and an AUC_{0-τ} from about 1 μM*h to about 50 μM*h; and

(2) an Iₘᵢₙ of about 50% or greater, or an I_{avg} of about 70% or greater.

The present invention also provides a method of treating cancer in a patient comprising administering to said patient a pharmaceutical composition comprising Compound 1, or a pharmaceutically acceptable salt thereof,

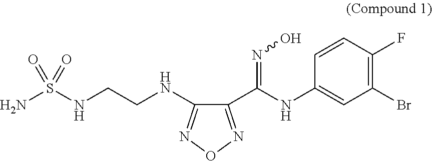 [SMILES: NS(=O)(=O)NCCNc1nonc1C(=NO)Nc1ccc(F)c(Br)c1]

and one or more excipients, wherein the treating comprises a dosage regimen comprising from about 25 mg to about 700 mg on a free base basis Compound 1, or a pharmaceutically acceptable salt thereof, administered orally twice daily, which attains at steady state, a Cₘₐₓ from about 0.10 μM to about 10 μM, a Cₘᵢₙ from about 0.01 μM to about 2.0 μM, a Tₘₐₓ of about 1 h to about 6 h and an AUC_{0-τ} from about 1 μM*h to about 50 μM*h.

The present invention also provides a method of treating cancer in a patient comprising administering to said patient one or more oral pharmaceutical composition provided herein and a second agent such as one or more inhibitors of an immune checkpoint molecule.

### BRIEF DESCRIPTION OF THE DRAWINGS

FIG. 1 shows an XRPD pattern characteristic of Compound 1 crystalline form.

FIG. 2 shows a DSC thermogram characteristic of Compound 1 crystalline form.

FIG. 3 shows TGA data characteristic of Compound 1 crystalline form.

FIG. 4 shows a graph of Compound 1 plasma concentrations by dose following the first dose.

FIG. 5 shows a graph of Compound 1 plasma concentrations by dose at steady state.

FIG. 6 shows a graph of Compound 1 plasma concentrations on C1D8 and C2D1.

FIG. 7 shows a graph of the dose proportional Cₘₐₓ of Compound 1 on C1D8 (all cohorts in part 1).

FIG. 8 shows a graph of the dose proportional AUC of Compound 1 on C1D8 (all cohorts in part 1).

FIG. 9 shows waterfall plots of projected percent IDO1 inhibition for various doses (N=58).

FIG. 10 shows a graph of Compound 1 plasma concentrations following the first dose between part 1 and part 2 in subjects receiving 100 mg BID.

FIG. 11 shows a graph of Compound 1 plasma concentrations at steady state (on C1D8) between part 1 and part 2 in subjects receiving 100 mg BID.

FIG. 12 shows a graph of Compound 1 trough plasma concentrations on C1D8 and C2D1 in subjects receiving 100 mg BID.

FIG. 13 shows a box plot of Compound 1 at steady state Cₘₐₓ for various tumor types.

FIG. 14 shows a box plot of Compound 1 at steady state AUCₜₐᵤ for various tumor types.

FIG. 15 shows waterfall plots of projected percent IDO1 inhibition at steady state.

### DETAILED DESCRIPTION

### Methods of Use

The present invention provides, inter alia, methods of treating cancer in a patient comprising administering to said patient one or more oral pharmaceutical compositions each comprising an IDO inhibitor, 4-({2-[(aminosulfonyl)amino]ethyl}amino)-N-(3-bromo-4-fluorophenyl)-N′-hydroxy-1,2,5-oxadiazole-3-carboximidamide (Compound 1), or a pharmaceutically acceptable salt thereof, and one or more excipients, wherein said one or more pharmaceutical compositions provide a certain pharmacokinetic profile of the compound that is useful in the treatment of disorders such as cancers. The structure of Compound 1 is depicted below.

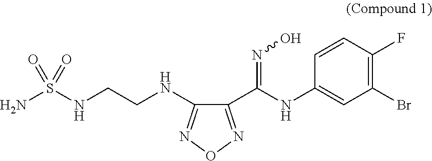 [SMILES: NS(=O)(=O)NCCNc1nonc1C(=NO)Nc1ccc(F)c(Br)c1]

A bond in a structure diagram represented by a wavy line [symbol] is intended to indicate that the structure represents the cis or the trans isomer, or a mixture of the cis and trans isomers in any proportion.

Pharmacokinetics (PK) allows those skilled in the art to monitor the fate of a drug from the moment that it is administered up to the point at which it is completely eliminated from the body. Pharmacokinetics describes how the body affects a specific drug after administration through the mechanisms of absorption and distribution, as well as the chemical changes of the substance in the body, and the effects and routes of excretion of the metabolites of the drug. Pharmacokinetic properties of drugs may be affected by elements such as the site of administration, formulation, solubility profile, fed/fast condition and the dose of administered drug, which may affect the absorption rate. Clinical PK monitoring is generally through determination of plasma concentrations because these data are reliable and can easily be obtained. Determining a drug's plasma concentration can help narrow the therapeutic range (e.g., difference between toxic and therapeutic concentrations) to reduce or minimize any side effects that the drug may have due to over-dosing.

Compound 1 as described herein is formulated in compositions that can be administered to a subject such as a human subject to achieve the desired PK profile effective in the treatment of cancers. The dosage regimen (e.g., Compound 1 is administered twice daily) can attain at steady state, an Iₘᵢₙ of about 50% or greater, or an I_{avg} of about 70% or greater, which can be effective in treating various cancers. Generally, following oral dose administration of compositions of the invention in the fasted state, the peak plasma concentration of Compound 1 is typically attained at 2 hours post-dose. Compound 1 is eliminated with a geometric mean terminal disposition half-life of 2.9 hours. It has been shown in the examples provided herein that increases in Compound 1 Cₘₐₓ and AUC_{0-τ} are less than proportional to dose. A high-fat meal delayed Compound 1 median Tₘₐₓ by 4 hours but does not cause clinically significant change in Compound 1 plasma exposures and thus, Compound 1 may be dosed without regard to food.

In vivo, it is believed that the primary pathway of Compound 1 clearance is via the glucuronidation in the liver. Enterohepatic circulation (EHC) occurs by biliary excretion and intestinal reabsorption of a drug, often with hepatic conjugation and intestinal deconjugation (Dobrinska, J Clin Pharmacol, 1989; 29:577-580). Without wishing to be bound by a particular theory, based on the glucuronide being the major metabolite of Compound 1, it is believed that EHC is involved in the disposition of Compound 1. Although the mean plasma concentration profiles of Compound 1 did not exhibit obvious patterns of secondary peaks (see e.g., Example 2), there were more than a few individual subjects who showed poorly defined plasma concentration peaks, secondary spiking in the concentration-time profiles, or otherwise unusually slow decline in Compound 1 plasma concentrations, particularly following the repeat doses. A prolonged Tₘₐₓ, however, would be consistent with a meal-stimulated excretion of bile into the small intestine which triggers EHC for Compound 1. Using a 1-compartment PK model of Compound 1 that fits the observed mean CL/F, Vz/F and Tₘₐₓ values, a simulation for BID dosing suggests that AUC_{0-τ} should accumulate by 8% at the steady-state, significantly less than 33% increase in AUC_{0-τ} as observed in Example 2, indicating compound sequestration beyond linear systemic accumulation. For compounds that undergo significant EHC, systemic accumulation tends to be under-predicted using observed t_{1/2} values, and calculation of “effective” t_{1/2} based on accumulation may be more meaningful (Dobrinska, J Clin Pharmacol, 1989; 29:577-580). The accumulation ratio based 10 on AUC suggests the “effective” t_{1/2} of about 6 hrs. Therefore, based on these observations, it is believed that EHC is involved in the disposition of Compound 1.

In some embodiments, provided herein is a method of treating cancer in a patient comprising administering to said patient a pharmaceutical composition comprising Compound 1, or a pharmaceutically acceptable salt thereof, and one or more excipients, wherein the treating comprises a dosage regimen comprising from about 25 mg to about 700 mg on a free basis of Compound 1, or a pharmaceutically acceptable salt thereof, administered orally twice daily.

In some embodiments, provide herein is a method of treating cancer in a patient comprising administering to said patient a pharmaceutical composition comprising Compound 1, or a pharmaceutically acceptable salt thereof, and one or more excipients, wherein the treating comprises a dosage regimen which attains at steady state, an Iₘᵢₙ of about 50% or greater, or an I_{avg} of about 70% or greater.

In some embodiments, provided herein is a method of treating cancer in a patient comprising administering to said patient a pharmaceutical composition comprising Compound 1, or a pharmaceutically acceptable salt thereof, and one or more excipients, wherein the treating comprises a dosage regimen which attains at steady state:
(1) a Cₘₐₓ from about 0.10 μM to about 10 μM, a Cₘᵢₙ from about 0.01 μM to about 2.0 μM, a Tₘₐₓ of about 1 h to about 6 h and an AUC_{0-τ} from about 1 μM*h to about 50 μM*h; and
(2) an Iₘᵢₙ of about 50% or greater, or an I_{avg} of about 70% or greater.

In some embodiments, provided herein is a method of treating cancer in a patient comprising administering to said patient a pharmaceutical composition comprising Compound 1, or a pharmaceutically acceptable salt thereof, and one or more excipients, in combination with a pharmaceutical composition comprising an inhibitor of an immune checkpoint molecule and one or more excipients, wherein the treating comprises a dosage regimen which attains at steady state, an Iₘᵢₙ of about 50% or greater, or an I_{avg} of about 70% or greater.

In some embodiments, provided herein is a method of treating cancer in a patient comprising administering to said patient a pharmaceutical composition comprising Compound 1, or a pharmaceutically acceptable salt thereof, and one or more excipients, in combination with a pharmaceutical composition comprising an inhibitor of an immune checkpoint molecule and one or more excipients, wherein the treating comprises a dosage regimen which attains at steady state:
(1) a Cₘₐₓ from about 0.10 μM to about 10 μM, a Cₘᵢₙ from about 0.01 μM to about 2.0 μM, a Tₘₐₓ of about 1 h to about 6 h and an AUC_{0-τ} from about 1 μM*h to about 50 μM*h; and
(2) an Iₘᵢₙ of about 50% or greater, or an I_{avg} of about 70% or greater.

In some embodiments, the inhibitor of an immune checkpoint molecule is pembrolizumab. In some embodiments, the dose regimen comprises from about 25 mg to about 300 mg on a free basis of Compound 1, or a pharmaceutically acceptable salt thereof, administered orally twice daily, and pembrolizumab administered every 21 days.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising from about 50 mg to about 300 mg on a free basis of Compound 1, or a pharmaceutically acceptable salt thereof, administered orally twice daily, wherein the dosage regimen attains a trough blood plasma concentration of a fasted individual at steady state equal to or greater than IC₅₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising from about 50 mg to about 300 mg on a free basis of Compound 1, or a pharmaceutically acceptable salt thereof, administered orally twice daily, wherein the dosage regimen attains an average blood plasma concentration of a fasted individual at steady state over the 12 hour interval that is equal to or greater than IC₉₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising from about 100 mg to about 300 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising from about 100 mg to about 300 mg on a free basis of Compound 1, or a pharmaceutically acceptable salt thereof, administered orally twice daily, wherein the dosage regimen attains an average blood plasma concentration of a fasted individual at steady state over the 12 hour interval that is equal to or greater than IC₉₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising from about 100 mg to about 300 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1 and an average blood plasma concentration of a fasted individual at steady state over the 12 hour interval that is equal to or greater than IC₉₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising about 100 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1 and an average blood plasma concentration of a fasted individual at steady state over the 12 hour interval that is equal to or greater than IC₉₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising about 200 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1 and an average blood plasma concentration of a fasted individual at steady state over the 12 hour interval that is equal to or greater than IC₉₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising about 300 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1 and an average blood plasma concentration of a fasted individual at steady state over the 12 hour interval that is equal to or greater than IC₉₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising from about 100 mg to about 300 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1 or an average blood plasma concentration of a fasted individual at steady state over the 12 hour interval that is equal to or greater than IC₉₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising about 100 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1 or an average blood plasma concentration of a fasted individual at steady state over the 12 hour interval that is equal to or greater than IC₉₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising about 200 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1 or an average blood plasma concentration of a fasted individual at steady state over the 12 hour interval that is equal to or greater than IC₉₀ at IDO1.

The present invention further provides a method of treating cancer in a patient comprising administering to said patient a pharmaceutical composition comprising Compound 1, or a pharmaceutically acceptable salt thereof, and one or more excipients, wherein the treating comprises a dosage regimen comprising about 300 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1 or an average blood plasma concentration of a fasted individual at steady state over the 12 hour interval that is equal to or greater than IC₉₀ at IDO1.

In some embodiments, provided herein is a method of treating cancer in a patient comprising administering to said patient a pharmaceutical composition comprising Compound 1, or a pharmaceutically acceptable salt thereof, and one or more excipients, wherein the treating comprises a dosage regimen comprising from about 25 mg to about 700 mg on a free basis of Compound 1, or pharmaceutically acceptable salt thereof, administered orally twice daily, which attains at steady state, a Cₘₐₓ from about 0.10 μM to about 10 μM, a Cₘᵢₙ from about 0.01 μM to about 2.0 μM, a Tₘₐₓ of about 1 h to about 6 h and an AUC_{0-τ} from about 1 μM*h to about 50 μM*h.

Wherein the term “dosage regimen” appears, the method may involve administering one or more pharmaceutical compositions to said patient. For example, in some embodiments, the methods provided herein comprises administering to a patient one or more pharmaceutical compositions to provide a dose of 25 mg to about 700 mg. For example, to achieve a dose of 400 mg, two compositions each comprising 200 mg on a free base basis of Compound 1, or a pharmaceutically acceptable salt thereof, may be administered to the patient.

In some embodiments, the Iₘᵢₙ is about 50% to about 80%, about 50% to about 70%, or about 50% to about 60%. For example, the Iₘᵢₙ is about 50% to about 60%.

In some embodiments, the I_{avg} is about 70% to about 90% or about 70% to about 80%. For example, the I_{avg} is about 70% to about 80%.

In some embodiments, the Cₘₐₓ is about 0.20 μM to about 8.0 μM, about 0.30 μM to about 7.0 μM, about 1.0 μM to about 7.0 μM, about 1.0 μM to about 6.0 μM, about 1.0 μM to about 5.0 μM, about 1.0 μM to about 4.0 μM, or about 1.0 μM to about 3.0 μM.

In some embodiments, the Cₘₐₓ is about 0.5 μM to about 7.0 μM, about 0.5 μM to about 6.0 μM, about 0.5 μM to about 5.0 μM, about 0.5 μM to about 4.0 μM, or about 0.5 μM to about 3.0 μM.

In some embodiments, the Cₘₐₓ is about 1.0 μM to about 3.0 μM. In some embodiment, the Cₘₐₓ is about 1.0 μM, about 2.0 μM, about 3.0 μM, about 4.0 μM, about 5.0 μM, about 6.0 μM, or about 7.0 μM. In some embodiments, Cₘₐₓ is about 0.9 μM to about 1.6 μM. In some embodiments, Cₘₐₓ is about 1.2 μM.

In some embodiments, the Cₘᵢₙ is about 0.01 μM to about 2.0 μM. In other embodiments, the Cₘᵢₙ is about 0.025 μM to about 0.5 μM.

In some embodiments, the Tₘₐₓ is about 1 h to about 4 h, about 1 h to about 3 h, or about 1 h to about 2 h. In some embodiments, the Tₘₐₓ is about 2 h to about 3 h. In some embodiments, the Tₘₐₓ is about 1 h to about 2 h. In some embodiments, the Tₘₐₓ is about 1 h, about 2 h, about 3 h, about 4 h, or about 5 h. In some embodiments, the Tₘₐₓ is about 2 h.

In some embodiments, the methods provided herein has an elimination half-life (t_{1/2}) about 2 h to about 4 h. In some embodiments, the t_{1/2} is about 2.5 h to about 4 h. In other embodiments, t_{1/2} is about 3.2 h.

In some embodiments, the AUC_{0-τ} is about 1 μM*h to about 40 μM*h, about 1 μM*h to about 36 μM*h, about 1 μM*h to about 30 μM*h, about 1 μM*h to about 20 μM*h, about 1 μM*h to about 10 μM*h, about 5 μM*h to about 15 μM*h, or about 5 μM*h to about 10 μM*h.

In some embodiments, the AUC_{0-τ} is about 4 μM*h to about 10 μM*h. In some embodiments, the AUC_{0-τ} is about 4 μM*h to about 6 μM*h. In some embodiments, the AUC_{0-τ} is about 4 μM*h to about 7 μM*h. In some embodiments, the AUC_{0-τ} is about 8 μM*h to about 10 μM*h. In some embodiments, the AUC_{0-τ} is about 4 μM*h, about 5 μM*h, about 6 μM*h, about 7 μM*h, about 8 μM*h, about 9 μM*h, or about 10 μM*h. In some embodiments, the AUC_{0-τ} is about 5 μM*h. In some embodiments, the AUC_{0-τ} is about 3.5 μM*h to about 8 μM*h. In some embodiments, the AUC_{0-τ} is about 5.5 μM*h.

In some embodiments, the dosage regiment comprises from about 50 mg to about 700 mg on a free basis of Compound 1, or pharmaceutically acceptable salt thereof. In some embodiments, the dosage regimen comprising about 25 mg to about 400 mg or about 50 mg to about 400 mg on a free base basis of Compound 1, or a pharmaceutically acceptable salt thereof, is administered twice daily

In some embodiments, the dosage regimen comprising about 25 mg to about 800 mg, about 25 mg to about 700 mg, about 25 mg to about 600 mg, about 25 mg to about 500 mg, about 25 mg to about 400 mg, about 25 mg to about 300 mg, about 25 mg to about 200 mg, about 25 mg to about 100 mg, about 100 to about 500 mg, or about 100 mg to about 400 mg on a free base basis of Compound 1, or a pharmaceutically acceptable salt thereof, is administered twice daily.

In some embodiments, the dosage regimen comprising about 25 mg to about 400 mg or about 50 mg to about 400 mg on a free base basis of Compound 1, or a pharmaceutically acceptable salt thereof, is administered twice daily.

In some embodiments, the dosage regimen comprising about 50 mg to about 400 mg on a free base basis of Compound 1, or a pharmaceutically acceptable salt thereof, is administered twice daily.

In some embodiments, the dosage regimen comprising about 200 mg to about 400 mg on a free base basis of Compound 1, or a pharmaceutically acceptable salt thereof, is administered twice daily.

In some embodiments, the dosage regimen comprising about 50 mg to about 200 mg on a free base basis of Compound 1, or a pharmaceutically acceptable salt thereof, is administered twice daily.

In some embodiments, the dosage regimen comprises about 50 mg to about 100 mg on a free basis of Compound 1, or a pharmaceutically acceptable salt thereof, administered orally twice daily.

In some embodiments, the dosage regimen comprises about 50 mg on a free basis of Compound 1, or a pharmaceutically acceptable salt thereof, administered orally twice daily.

In some embodiments, the dosage regimen comprises about 100 mg on a free basis of Compound 1, or a pharmaceutically acceptable salt thereof, administered orally twice daily.

In some embodiments, the dosage regimen comprising about 100 mg to about 700 mg on a free basis of Compound 1, or a pharmaceutically acceptable salt thereof, is administered orally twice daily.

In some embodiments, the dosage regimen comprising about 100 mg to about 400 mg on a free basis of Compound 1, or a pharmaceutically acceptable salt thereof, is administered orally twice daily.

In some embodiments, the dosage regimen comprising about 100 mg to about 300 mg on a free basis of Compound 1, or a pharmaceutically acceptable salt thereof, is administered orally twice daily.

In some embodiments, the dosage regimen comprising about 25 mg, about 50 mg, about 100 mg, about 200 mg, about 300 mg, about 400 mg, about 500 mg, about 600 mg, or about 700 mg on a free base basis of Compound 1, or a pharmaceutically acceptable salt thereof, is administered twice daily.

In some embodiments, the dosage regimen comprising about 25 mg, about 100 mg, or about 300 mg on a free base basis of Compound 1, or a pharmaceutically acceptable salt thereof, is administered twice daily.

In some embodiments, the dosage regimen comprising about 100 mg, about 200 mg, or about 300 mg on a free base basis of Compound 1, or a pharmaceutically acceptable salt thereof, is administered twice daily.

In some embodiments, the dosage regimen comprising about 100 mg or about 300 mg on a free base basis of Compound 1, or a pharmaceutically acceptable salt thereof, is administered twice daily.

In some embodiments, the dosage regimen comprising about 100 mg on a free base basis of Compound 1, or a pharmaceutically acceptable salt thereof, is administered twice daily.

In some embodiments, the dosage regimen comprising about 200 mg on a free base basis of Compound 1, or a pharmaceutically acceptable salt thereof, is administered twice daily.

In some embodiments, the dosage regimen comprising about 300 mg on a free base basis of Compound 1, or a pharmaceutically acceptable salt thereof, is administered twice daily.

In some embodiments, said one or more pharmaceutical compositions are administered twice-per-day (BID) to said patient. In some embodiments, said one or more pharmaceutical compositions are administered once-per-day (QD) to said patient. In some embodiments, said one or more pharmaceutical compositions are administered three times per day, four times per day, or five times per day to said patient.

In some embodiments, each composition suitable for oral administration. In some embodiments, each composition is formulated as a tablet, a capsule, a liquid form or an aqueous solution form. In some embodiments, each composition is formulated as a tablet. In some embodiments, multiple tablets are administered to achieve a desired dose. For example, a tablet of about 300 mg and a tablet of about 100 mg can be administered to the subject to achieve a dose about 400 mg. In some embodiments, multiple tablets are taken contemporaneously or sequentially.

In some embodiments, the dosage regimen comprising about 50 mg on a free base basis of Compound 1, or a pharmaceutically acceptable salt thereof, is administered twice daily, which attains, at steady state, a Cₘₐₓ of about 0.1 μM to about 1.0 μM or about 0.3 μM to about 1.3 μM, a Tₘₐₓ of about 2 h, and an AUC_{0-τ} of about 1 μM*h to about 3 μM*h.

In some embodiments, the dosage regimen comprising about 100 mg on a free base basis of Compound 1, or a pharmaceutically acceptable salt thereof, is administered twice-per-day which provides, at steady state, a Cₘₐₓ of about 0.5 μM to about 2.0 μM, Tₘₐₓ of about 2 h and an AUC_{0-τ} of about 4 μM*h to about 7 μM*h.

In some embodiments, the dosage regimen comprising about 300 mg on a free base basis of Compound 1, or a pharmaceutically acceptable salt thereof, is administered twice-per-day which provides, at steady state, a Cₘₐₓ of about 1.0 μM to about 3.0 μM, a Tₘₐₓ of about 2 and an AUC_{0-τ} of about 8 μM*h to about 10 μM*h.

In some embodiments, the dosage regimen comprising about 100 mg to about 300 mg on a free base basis of Compound 1, or a pharmaceutically acceptable salt thereof, is administered twice daily, which attains at steady state, an Iₘᵢₙ of about 50% or greater, or an I_{avg} of about 70% or greater.

In some embodiments, the dosage regimen comprising about 100 mg on a free base basis of Compound 1, or a pharmaceutically acceptable salt thereof, is administered twice daily, which attains at steady state, an Iₘᵢₙ of about 50% or greater, or an I_{avg} of about 70% or greater.

In some embodiments, the excipient is selected from lactose monohydrate, microcrystalline cellulose, povidone, croscarmellose sodium, colloidal silicon dioxide, and magnesium stearate

In some embodiments, lactose monohydrate is present in an amount about 20 wt % to about 35 wt % or about 24 wt % to about 32 wt % of a composition provided herein. In some embodiments, lactose monohydrate is present in an amount about 24 wt % to about 29 wt %.

In some embodiments, lactose monohydrate is present in an amount about 24 wt %, about 25 wt %, about 26 wt %, about 27 wt %, about 28 wt %, about 29 wt %, about 30 wt %, about 31 wt %, or about 32 wt %. In some embodiments, lactose monohydrate is present in an amount about 25 wt %, about 29 wt %, about 31 wt %, or about 32 wt %. In some embodiments, lactose monohydrate is present in an amount about 24.5 wt %, about 28.8 wt %, about 30.75 wt %, or about 32.1 wt %.

In some embodiments, microcrystalline cellulose is present in an amount about 20 wt % to about 35 wt % or about 22 wt % to about 33 wt % of a composition provided herein. In some embodiments, microcrystalline cellulose is present in an amount about 22 wt %, about 23 wt %, about 24 wt %, about 25 wt %, about 26 wt %, about 27 wt %, about 28 wt %, about 29 wt %, about 30 wt %, about 31 wt %, about 32 wt %, or about 33 wt %. In some embodiments, microcrystalline cellulose is present in an amount about 22 wt %, about 24 wt %, or about 33 wt %. In some embodiments, microcrystalline cellulose is present in an amount about 22.0 wt %, about 24.2 wt %, or about 32.8 wt %.

In some embodiments, povidone is present in an amount about 0.5 wt % to about 1.0 wt % of a composition provided herein. In some embodiments, povidone is present in an amount about 0.8 wt %.

In some embodiments, croscarmellose sodium is present in an amount about 1.0 wt % to about 10.0 wt % of a composition provided herein. In some embodiments, croscarmellose sodium is present in an amount about 3.2 wt % or about 9.6 wt %. In some embodiments, croscarmellose sodium is present in an amount about 3.2 wt %.

In some embodiments, colloidal silicon dioxide is present in an amount about 0.1 wt % to about 1.0 wt % of a composition provided herein. In some embodiments, colloidal silicon dioxide is present in an amount about 0.5 wt % to 1.0 wt %. In some embodiments, colloidal silicon dioxide is present in an amount about 0.6 wt % or about 0.7 wt %.

In some embodiments, magnesium stearate is present in an amount about 0.1 wt % to about 1.0 wt % of a composition provided herein. In some embodiments, magnesium stearate is present in an amount about 0.6 wt %.

In some embodiments, the present invention provides a method of treating cancer in a patient comprising administering to said patient one or more oral pharmaceutical compositions each comprising 25 mg Compound 1, or a pharmaceutically acceptable salt thereof, and one or more excipients selected from about 31 wt % to about 32 wt % of lactose monohydrate, about 24 wt % to about 33 wt % of microcrystalline cellulose, about 0.5 wt % to about 1.0 wt % of povidone, about 1.0 wt % to about 10.0 wt % of croscarmellose sodium, about 0.1 wt % to about 1.0 wt % of colloidal silicon dioxide, and about 0.1 wt % to about 1.0 wt % of magnesium stearate.

In some embodiments, the present invention provides a method of treating cancer in a patient comprising administering to said patient one or more oral pharmaceutical compositions each comprising 100 mg Compound 1, or a pharmaceutically acceptable salt thereof, and one or more excipients selected from about 31 wt % to about 32 wt % of lactose monohydrate, about 24 wt % to about 33 wt % of microcrystalline cellulose, about 0.1 wt % to about 1.0 wt % of povidone, about 1.0 wt % to about 10.0 wt % of croscarmellose sodium, about 0.1 wt % to about 1.0 wt % colloidal silicon dioxide, and about 0.1 wt % to about 1.0 wt % of magnesium stearate.

In some embodiments, the present invention provides a method of treating cancer in a patient comprising administering to said patient one or more oral pharmaceutical compositions each comprising 300 mg Compound 1, or a pharmaceutically acceptable salt thereof, and one or more excipients selected from about 24 wt % to about 29 wt % of lactose monohydrate, about 22 wt % to about 33 wt % of microcrystalline cellulose, about 0.1 wt % to about 1.0 wt % of povidone, about 1.0 wt % to about 10.0 wt % of croscarmellose sodium, about 0.5 wt % to about 1.0 wt % colloidal silicon dioxide, and about 0.1 wt % to about 0.6 wt % of magnesium stearate.

In some embodiments, the present invention provides method of treating melanoma in a patient comprising administering to said patient a pharmaceutical composition comprising Compound 1, or a pharmaceutically acceptable salt thereof, and one or more excipients, wherein the treating comprises a dosage regimen comprising from about 50 mg to about 700 mg on a free basis of Compound 1, or a pharmaceutically acceptable salt thereof, administered orally twice daily, and one or more inhibitors of an immune checkpoint molecule.

In some embodiments, the present invention provides method of treating melanoma in a patient comprising administering to said patient a pharmaceutical composition comprising Compound 1, or a pharmaceutically acceptable salt thereof, and one or more excipients, wherein the treating comprises a dosage regimen comprising from about 50 mg to about 300 mg on a free basis of Compound 1, or a pharmaceutically acceptable salt thereof, administered orally twice daily, and pembrolizumab administered every three weeks.

In some embodiments, the present invention provides method of treating melanoma in a patient comprising administering to said patient a pharmaceutical composition comprising Compound 1, or a pharmaceutically acceptable salt thereof, and one or more excipients, wherein the treating comprises a dosage regimen comprising from about 100 mg to about 300 mg on a free basis of Compound 1, or a pharmaceutically acceptable salt thereof, administered orally twice daily, and one or more inhibitors of an immune checkpoint molecule.

In some embodiments, the present invention provides method of treating melanoma in a patient comprising administering to said patient a pharmaceutical composition comprising Compound 1, or a pharmaceutically acceptable salt thereof, and one or more excipients, wherein the treating comprises a dosage regimen comprising from about 100 mg to about 300 mg on a free basis of Compound 1, or a pharmaceutically acceptable salt thereof, administered orally twice daily, and pembrolizumab administered every three weeks.

In some embodiments, the present invention provides method of treating melanoma in a patient comprising administering to said patient a pharmaceutical composition comprising Compound 1, or a pharmaceutically acceptable salt thereof, and one or more excipients, in combination with a pharmaceutical composition comprising pembrolizumab and one or more excipients, wherein the treating comprises a dosage regimen comprising from about 25 mg to about 300 mg on a free basis of Compound 1, or a pharmaceutically acceptable salt thereof, administered orally twice daily, and pembrolizumab administered every three weeks.

In some embodiments, the present invention is directed to a method of preparing a pharmaceutical composition as described herein, comprising mixing Compound 1, or a pharmaceutically acceptable salt thereof, with one or more excipients selected from lactose monohydrate, microcrystalline cellulose, povidone, croscarmellose sodium, colloidal silicon dioxide, and magnesium stearate.

In some embodiments, the patient is in a fasted state. The term “fasted” refers to prior to administration of a composition provided herein, the patient has been fasting for at least 2 hours and remained fasted for 1 hour after dose administration.

Compound 1 can be prepared according the procedures in U.S. Pat. No. 8,088,803 and US Publication No. 2015/0133674, the entireties of which are incorporated herein by reference.

Compound 1 can exist in various solid forms. As used herein “solid form” is meant to refer to a solid characterized by one or more properties such as, for example, melting point, solubility, stability, crystallinity, hygroscopicity, water content, TGA features, DSC features, DVS features, XRPD features, etc. Solid forms, for example, can be amorphous, crystalline, or mixtures thereof.

Different crystalline solid forms typically have different crystalline lattices (e.g., unit cells) and, usually as a result, have different physical properties. In some instances, different crystalline solid forms have different water or solvent content. The different crystalline lattices can be identified by solid state characterization methods such as by X-ray powder diffraction (XRPD). Other characterization methods such as differential scanning calorimetry (DSC), thermogravimetric analysis (TGA), dynamic vapor sorption (DVS), and the like further help identify the solid form as well as help determine stability and solvent/water content.

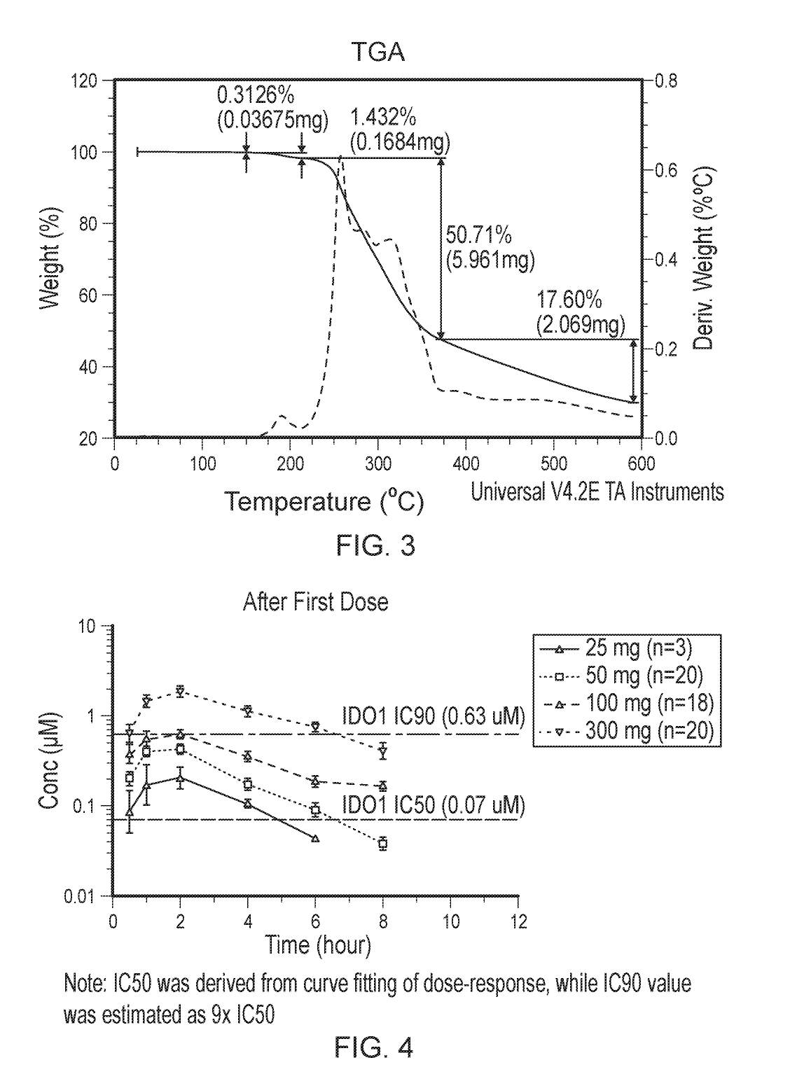

In some embodiments, the solid form is a crystalline solid. In some embodiments, Compound 1 is the crystalline solid as described in U.S. Pat. No. 8,088,803. In some embodiments, the solid form is substantially anhydrous (e.g., contains less than about 1% water, less than about 0.5% water, less than about 1.5% water, less than about 2% water). For example, the water content is determined by Karl Fischer titration. In some embodiments, the solid form is characterized by a melting point of, or a DSC endotherm centered at, about 162 to about 166° C. In some embodiments, the solid form is characterized by a melting point of, or a DSC endotherm centered at, about 164° C. In some embodiments, the solid form has a DSC thermogram substantially as shown in FIG. 2. In some embodiments, the solid form has a weight loss of 0.3%, heating from 20° C. to 150° C. at a heating rate of 10° C./min. See thermogravimetric analysis (TGA) (FIG. 3) using a TA Instrument Q500.

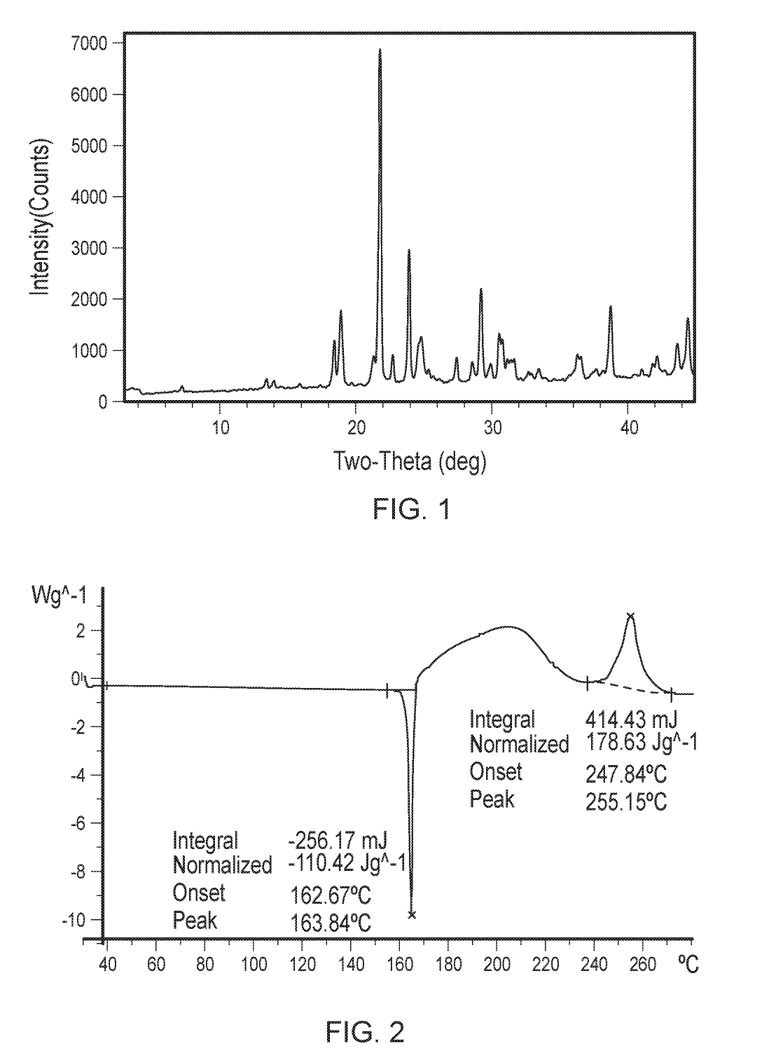

In further embodiments, the solid form has at least one, two or three XRPD peaks, in terms of 2-theta, selected from about 18.4°, about 18.9°, about 21.8°, about 23.9°, about 29.2°, and about 38.7°. In further embodiments, the solid form has an XRPD pattern substantially as shown in FIG. 1.

In some embodiments, the crystalline form has one or more of the peaks from the list of 2-theta peaks provided in table below.

| 2-Theta | Height | H % |
|---|---|---|
| 3.9 | 74 | 1.1 |
| 7.2 | 119 | 1.8 |
| 13.4 | 180 | 2.8 |
| 14.0 | 150 | 2.3 |
| 15.9 | 85 | 1.3 |
| 18.4 | 903 | 13.9 |
| 18.9 | 1469 | 22.7 |
| 21.3 | 519 | 8 |
| 21.8 | 6472 | 100 |
| 22.7 | 516 | 8 |
| 23.9 | 2515 | 38.9 |
| 24.8 | 804 | 12.4 |
| 25.3 | 182 | 2.8 |
| 27.4 | 476 | 7.4 |
| 28.6 | 354 | 5.5 |
| 29.2 | 1767 | 27.3 |
| 29.9 | 266 | 4.1 |
| 30.6 | 773 | 11.9 |
| 31.2 | 379 | 5.8 |
| 31.6 | 291 | 4.5 |
| 32.7 | 144 | 2.2 |
| 33.5 | 221 | 3.4 |
| 36.4 | 469 | 7.2 |
| 37.6 | 152 | 2.3 |
| 38.7 | 1381 | 21.3 |
| 41.0 | 153 | 2.4 |
| 42.1 | 382 | 5.9 |
| 43.6 | 527 | 8.1 |
| 44.4 | 1080 | 16.7 |

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 4% of the maximum peak height/intensity. Moreover, instrument variation and other factors can affect the 2-theta values. Thus, peak assignments, such as those reported herein, can vary by plus or minus about 0.2° (2-theta), and the term “substantially” as used in the context of XRPD herein is meant to encompass the above-mentioned variations.

In the same way, temperature readings in connection with DSC, TGA, or other thermal experiments can vary about +3° C. depending on the instrument, particular settings, sample preparation, etc. Accordingly, a crystalline form reported herein having a DSC thermogram “substantially” as shown in any of the Figures is understood to accommodate such variation.

The term “Cₘₐₓ” refers to the maximum plasma concentration of Compound 1. The term “Cₘᵢₙ” refers to the minimum plasma concentration of Compound 1. These values are taken directly from the observed plasma concentration data.

The term “Tₘₐₓ” refers to the time at which Cₘₐₓ is observed. The value is taken directly from the observed plasma concentration data.

The term “t_{1/2}” refers to the time taken for the plasma concentration of Compound 1 to fall by half its original value.

The term “AUC” refers to the area under the curve in a plot of concentration of Compound 1 in the plasma against time. For example, AUC₀₋₂₄ₕ refers to the area under the curve in a plot of concentration of Compound 1 in the plasma from time 0 to 24 hour.

The term “AUC_{0-∞}” refers to the area under the curve in a plot of concentration of Compound 1 in the plasma extrapolated to infinity.

The term “AUC₀₋ₜ” refers to the area under the plasma concentration-time curve from time 0 to the last time point with a quantifiable plasma concentration, usually about 12-36 hours.

As used herein, “AUC_{0-τ}” refers to the area under the plasma concentration-time curve from time 0 to the time of the next dose.

The term “Cl/F” refers to oral clearance.

The term “steady state” refers to the state when the overall intake of a drug is close in dynamic equilibrium with its elimination.

The inhibition of IDO1 using Compound 1 was calculated using the equation: Conc/(Conc+EC₅₀)*100(%). For example, when Conc=0 then inhibition=0, and when Conc approaches EC₅₀, then inhibition approaches 50%. The plasma concentration was measured by a validated GLP LC/MS/MS method with a linear range of 0.020 to 20.0 μM.

The term “Iₘₐₓ” refers to the maximum percentage of the calculated IDO inhibition across all the PK time points. Iₘₐₓ is the maximum or highest percentage of IDO inhibition between the time when the drug is administered to its trough (e.g., the lowest concentration of the drug that is present in the subject). For example, in a twice-daily administration, Iₘₐₓ refers to the highest percentage of IDO inhibition during the period between 0 hour (pre-dose) and 12^{th} hour after dosing.

The term “Iₘᵢₙ” refers to the minimum percentage of the calculated IDO inhibition across all the PK time points. Iₘᵢₙ is the percentage of IDO inhibition at trough (e.g., generally at the 12^{th} hour in a twice-daily administration). For example, Iₘᵢₙ>50 refers to IDO inhibition is 50% or greater at trough (e.g. at the 12^{th} hour).

The term “I_{avg}” refers to the average percentage of IDO inhibition during the period from which the drug is administered to trough. It is calculated as the area under the inhibition curve over time (AUC) (calculated using a linear trapezoidal method) divided by the dosing interval (e.g., 12 hours for BID dosing).

The calculated Iₘₐₓ, Iₘᵢₙ and I_{avg} values of each subject were summarized as mean±standard deviation (geometric mean) standard statistical calculations for every dose group such as 25 mg QD, 50 mg QD, etc.

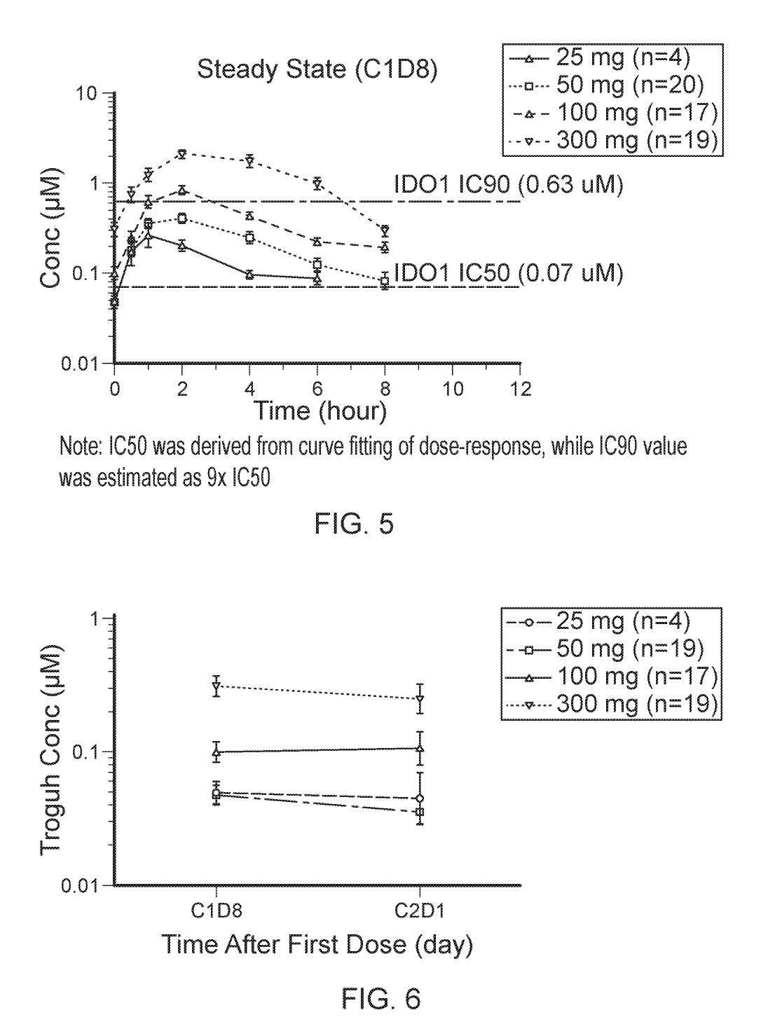

The term “IC₅₀” refers to the concentration of Compound 1 where the response is reduced by half. This value can be derived from curve fitting of dose-response. FIGS. 4 and 5 show the IC₅₀ of various doses of Compound 1 after first dose and at steady state. IC₅₀ for IDO1 was calculated as 70 nM in a population pharmacokinetic-pharmacodynamic analysis of time-matched Compound 1, tryptophan and kynurenine plasma concentrations (see Example 3 for more details), which was consistent with both in vitro results in human whole blood (125±26 nM [n=5], Table 1 in Investigator's Brochure Version 7) and clinical results (127 nM [n=284, all available data] and 90 nM [n=216, data from BID dosing only].

The term “IC₉₀” refers to the concentration of Compound 1 that is estimated by nine times the value of IC₅₀.

In some embodiments, the term “about” refers to plus or minus 10% of the value. A skilled person in the art would know that the values presented herein can vary due to the conditions of the experiments such as variability in data collection or instruments.

Compound 1 described herein also includes tautomeric forms. Tautomeric forms result from the swapping of a single bond with an adjacent double bond together with the concomitant migration of a proton.

Compound 1 described herein also includes all isotopes of atoms occurring in the intermediates or final compounds. Isotopes include those atoms having the same atomic number but different mass numbers. For example, isotopes of hydrogen include tritium and deuterium.

In some embodiments, Compound 1 and salts thereof are substantially isolated. By “substantially isolated” is meant that the compound is at least partially or substantially separated from the environment in which it was formed or detected. Partial separation can include, for example, a composition enriched in Compound 1. Substantial separation can include compositions containing at least about 50%, at least about 60%, at least about 70%, at least about 80%, at least about 90%, at least about 95%, at least about 97%, or at least about 99% by weight of Compound 1, or salt thereof. Methods for isolating compounds and their salts are routine in the art.

The present invention also includes salts of Compound 1 described herein. As used herein, “salts” refers to derivatives of the disclosed compound wherein the parent compound is modified by converting an existing acid or base moiety to its salt form. Examples of salts include, but are not limited to, mineral acid (such as HCl, HBr, H2SO4) or organic acid (such as acetic acid, benzoic acid, trifluoroacetic acid) salts of basic residues such as amines; alkali (such as Li, Na, K, Mg, Ca) or organic (such as trialkylammonium) salts of acidic residues such as carboxylic acids; and the like. The salts of the present invention can be synthesized from the parent compound which contains a basic or acidic moiety by conventional chemical methods. Generally, such salts can be prepared by reacting the free acid or base forms of Compound 1 with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred.

The “pharmaceutically acceptable salts” of the present invention include a subset of the “salts” described above which are, conventional non-toxic salts of the parent compound formed, for example, from non-toxic inorganic or organic acids. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In some embodiments, the pharmaceutical compositions described herein comprises one or more excipients or pharmaceutically acceptable carriers. These compositions can be prepared in a manner well known in the pharmaceutical art, and can be administered by a variety of routes, depending upon whether local or systemic treatment is desired and upon the area to be treated.

In some embodiments, the pharmaceutical compositions described herein is suitable for oral administration.

In some embodiments, in making the compositions provided herein, Compound 1 is mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In some embodiments, the pharmaceutical compositions described herein is in the form of tablets.

In preparing a formulation, Compound 1 can be milled to provide the appropriate particle size prior to combining with the other ingredients. In some embodiments, Compound 1 can be milled to a particle size of less than 200 mesh. In some embodiments, the particle size can be adjusted by milling to provide a substantially uniform distribution in the formulation, e.g. about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions provided herein can be formulated so as to provide quick, sustained or delayed release of the active ingredient after administration to the patient by employing procedures known in the art.

The compositions can be formulated in a unit dosage form. The term “unit dosage forms” refers to physically discrete units suitable as unitary dosages for human subjects and other mammals, each unit containing a predetermined quantity of Compound 1 calculated to produce the desired therapeutic effect (e.g., the desired PK profile), in association with a suitable pharmaceutical excipient.

In certain embodiments, for preparing solid compositions such as tablets, Compound is mixed with a pharmaceutical excipient to form a solid pre-formulation composition containing a homogeneous mixture of Compound 1. When referring to these pre-formulation compositions as homogeneous, Compound 1 is typically dispersed evenly throughout the composition so that the composition can be readily subdivided into equally effective unit dosage forms such as tablets, pills and capsules. This solid pre-formulation is then subdivided into unit dosage forms.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions described herein can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

In some embodiments, compositions described herein are sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of Compound can vary according to, for example, the particular use for which the treatment is made, the manner of administration of the compound, the health and condition of the patient, and the judgment of the prescribing physician. In some embodiments, the dosage of Compound 1 is determined by achieving a PK profile as described herein (e.g., certain Cₘₐₓ, Cₘᵢₙ, Tₘₐₓ, and/or AUC values). The proportion or concentration of Compound 1 in a pharmaceutical composition can vary depending upon a number of factors including dosage, chemical characteristics (e.g., hydrophobicity), and the route of administration. Compound 1 can also be formulated in combination with one or more additional active ingredients which can include any pharmaceutical agent such as anti-viral agents, vaccines, antibodies, immune enhancers, immune suppressants, anti-inflammatory agents and the like.

Compound 1 as described herein can inhibit activity of the enzyme indoleamine-2,3-dioxygenase (IDO or IDO1). For example, Compound 1 can be used to inhibit activity of IDO in cell or in an individual in need of modulation of the enzyme by administering an inhibiting amount of Compound 1.

The present invention further provides methods of inhibiting the degradation of tryptophan in a system containing cells expressing IDO such as a tissue, living organism, or cell culture. In some embodiments, the present invention provides methods of altering (e.g., increasing) extracellular tryptophan levels in a mammal by administering an effective amount of Compound 1 or compositions provided herein. Methods of measuring tryptophan levels and tryptophan degradation are routine in the art.

The present invention further provides methods of inhibiting immunosuppression such as IDO-mediated immunosuppression in a patient by administering to the patient an effective amount of a compound or composition recited herein. IDO-mediated immunosuppression has been associated with, for example, cancers, tumor growth, metastasis, viral infection, viral replication, etc.

The present invention further provides methods of treating diseases associated with activity or expression, including abnormal activity and/or overexpression, of IDO in an individual (e.g., patient) by administering to the individual in need of such treatment a therapeutically effective amount or dose of a compound of the present invention or a pharmaceutical composition thereof. Example diseases can include any disease, disorder or condition that is directly or indirectly linked to expression or activity of the IDO enzyme, such as over expression or abnormal activity. An IDO-associated disease can also include any disease, disorder or condition that can be prevented, ameliorated, or cured by modulating enzyme activity. Examples of IDO-associated diseases include cancer, viral infection such as HIV infection, HCV infection, depression, neurodegenerative disorders such as Alzheimer's disease and Huntington's disease, trauma, age-related cataracts, organ transplantation (e.g., organ transplant rejection), and autoimmune diseases including asthma, rheumatoid arthritis, multiple sclerosis, allergic inflammation, inflammatory bowel disease, psoriasis and systemic lupus erythematosus. Example cancers treatable by the methods herein include colon cancer, pancreatic cancer, breast cancer, prostate cancer, lung cancer, brain cancer, ovarian cancer, cervical cancer, testicular cancer, renal cancer, head and neck cancer, and lymphoma, leukemia. In some embodiments, the cancer is solid tumor. In some embodiments, the cancer is melanoma, non-small-cell lung carcinoma, genitourinary cancer (e.g., transitional cell carcinoma of the genitourinary (GU) tract), renal cell cancer, triple negative breast cancer (TNBC), adenocarcinoma of the endometrium, squamous cell carcinoma of the head and neck (SCCHN), endometrial cancer, gastric cancer, pancreatic ductal adenocarcinoma, diffuse large B-cell lymphoma (DLBCL), or ovarian cancer (OC). In some embodiments, the cancer is melanoma. Compound 1 can also be useful in the treatment of obesity and ischemia.

In some embodiments, the present invention is directed to a method of treating cancer in a subject comprising administering to the subject a pharmaceutical composition described herein.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” the IDO enzyme with Compound 1 includes the administration of Compound 1 to an individual or patient, such as a human, having IDO, as well as, for example, introducing Compound 1 into a sample containing a cellular or purified preparation containing the IDO enzyme.

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans.

As used herein, the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” refers to preventing a disease, condition or disorder in an individual who may be predisposed to the disease, condition or disorder but does not yet experience or display the pathology or symptomatology of the disease.

### Combination Therapy

One or more additional pharmaceutical agents or treatment methods such as, for example, anti-viral agents, chemotherapeutics or other anti-cancer agents, immune enhancers, immunosuppressants, radiation, anti-tumor and anti-viral vaccines, cytokine therapy (e.g., IL2, GM-CSF, etc.), and/or tyrosine kinase inhibitors can be used in combination with Compound 1 for treatment of IDO-associated diseases, disorders or conditions. The agents can be combined with Compound 1 in a single dosage form, or the agents can be administered simultaneously or sequentially as separate dosage forms.

Suitable antiviral agents contemplated for use in combination with Compound 1 can comprise nucleoside and nucleotide reverse transcriptase inhibitors (NRTIs), non-nucleoside reverse transcriptase inhibitors (NNRTIs), protease inhibitors and other antiviral drugs.

Example suitable NRTIs include zidovudine (AZT); didanosine (ddl); zalcitabine (ddC); stavudine (d4T); lamivudine (3TC); abacavir (1592U89); adefovir dipivoxil [bis(POM)-PMEA]; lobucavir (BMS-180194); BCH-10652; emitricitabine [(−)-FTC]; beta-L-FD4 (also called beta-L-D4C and named beta-L-2′, 3′-dicleoxy-5-fluoro-cytidene); DAPD, ((−)-beta-D-2,6,-diamino-purine dioxolane); and lodenosine (FddA). Typical suitable NNRTIs include nevirapine (BI-RG-587); delaviradine (BHAP, U-90152); efavirenz (DMP-266); PNU-142721; AG-1549; MKC-442 (1-(ethoxy-methyl)-5-(1-methylethyl)-6-(phenylmethyl)-(2,4(1H,3H)-pyrimidinedione); and (+)-calanolide A (NSC-675451) and B. Typical suitable protease inhibitors include saquinavir (Ro 31-8959); ritonavir (ABT-538); indinavir (MK-639); nelfnavir (AG-1343); amprenavir (141W94); lasinavir (BMS-234475); DMP-450; BMS-2322623; ABT-378; and AG-1 549. Other antiviral agents include hydroxyurea, ribavirin, IL-2, IL-12, pentafuside and Yissum Project No. 11607.

Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethio-phosphoramine, busulfan, carmustine, lomustine, streptozocin, dacarbazine, and temozolomide.

In the treatment of melanoma, suitable agents for use in combination with the compounds of the present invention include: dacarbazine (DTIC), optionally, along with other chemotherapy drugs such as carmustine (BCNU) and cisplatin; the “Dartmouth regimen,” which consists of DTIC, BCNU, cisplatin and tamoxifen; a combination of cisplatin, vinblastine, and DTIC; or temozolomide. Compounds according to the invention may also be combined with immunotherapy drugs, including cytokines such as interferon alpha, interleukin 2, and tumor necrosis factor (TNF) in the treatment of melanoma.

Compound 1 may also be used in combination with vaccine therapy in the treatment of melanoma. Antimelanoma vaccines are, in some ways, similar to the anti-virus vaccines which are used to prevent diseases caused by viruses such as polio, measles, and mumps. Weakened melanoma cells or parts of melanoma cells called antigens may be injected into a patient to stimulate the body's immune system to destroy melanoma cells.

Melanomas that are confined to the arms or legs may also be treated with Compound 1 using a hyperthermic isolated limb perfusion technique. This treatment protocol temporarily separates the circulation of the involved limb from the rest of the body and injects high doses of chemotherapy into the artery feeding the limb, thus providing high doses to the area of the tumor without exposing internal organs to these doses that might otherwise cause severe side effects. Usually the fluid is warmed to 102° to 104° F. Melphalan is the drug most often used in this chemotherapy procedure. This can be given with another agent called tumor necrosis factor (TNF) (see section on cytokines).

Suitable chemotherapeutic or other anti-cancer agents include, for example, antimetabolites (including, without limitation, folic acid antagonists, pyrimidine analogs, purine analogs and adenosine deaminase inhibitors) such as methotrexate, 5-fluorouracil, floxuridine, cytarabine, 6-mercaptopurine, 6-thioguanine, fludarabine phosphate, pentostatine, and gemcitabine.

Suitable chemotherapeutic or other anti-cancer agents further include, for example, certain natural products and their derivatives (for example, vinca alkaloids, antitumor antibiotics, enzymes, lymphokines and epipodophyllotoxins) such as vinblastine, vincristine, vindesine, bleomycin, dactino-mycin, daunorubicin, doxorubicin, epirubicin, idarubicin, ara-C, paclitaxel (TAXOL™), mithramycin, deoxycoformycin, mitomycin-C, L-asparaginase, interferons (especially IFN-α), etoposide, and teniposide.

Other cytotoxic agents include navelbene, CPT-11, anastrazole, letrazole, capecitabine, reloxafine, cyclophosphamide, ifosamide, and droloxafine.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Other anti-cancer agent(s) include antibody therapeutics such as trastuzumab (Herceptin), antibodies to costimulatory molecules such as CTLA-4, 4-1BB and PD-1, or antibodies to cytokines (IL-10, TGF-3, etc.).

In some embodiments, Compound 1 provided herein can be used in combination with one or more immune checkpoint inhibitors for the treatment of cancer as described herein. In one embodiment, the combination with one or more immune checkpoint inhibitors as described herein can be used for the treatment of melanoma. Exemplary immune checkpoint inhibitors include inhibitors against immune checkpoint molecules such as CD27, CD28, CD40, CD122, OX40, GITR, CD137, ICOS, A2AR, B7-H3, B7-H4, BTLA, CTLA-4, LAG3, TIM3, VISTA, PD-1, PD-L1 and PD-L2. In some embodiments, Compound 1 provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, or AMP-224. In some embodiments, the anti-PD-1 monoclonal antibody is nivolumab or pembrolizumab. In some embodiments, the anti-PD1 antibody is pembrolizumab. The amount of pembrolizumab can be about 2 mg/kg. In some examples, pembrolizumab is administered at a frequency of about every three weeks.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the inhibitor of an immune checkpoint molecule is an inhibitor of LAG3, e.g., an anti-LAG3 antibody. In some embodiments, the anti-LAG3 antibody is BMS-986016.

Other anti-cancer agents also include those that block immune cell migration such as antagonists to chemokine receptors, including CCR2 and CCR4.

Other anti-cancer agents also include those that augment the immune system such as adjuvants or adoptive T cell transfer.

Anti-cancer vaccines include dendritic cells, synthetic peptides, DNA vaccines and recombinant viruses.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, N.J.), the disclosure of which is incorporated herein by reference as if set forth in its entirety.

### Kits

The present invention also includes pharmaceutical kits useful, for example, in the treatment or prevention of IDO-associated diseases or disorders, obesity, diabetes and other diseases referred to herein which include one or more containers containing a pharmaceutical composition described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

### Example 1. Formulations of Compound 1

Compound 1 is formulated as 25 mg, 100 mg, and 300 mg tablets. Croscarmellose sodium content is reduced from 9.6 wt % in Formulations 1 and 3 to 3.2 wt % in Formulations 2 and 4. This change was made to bring the level of croscarmellose sodium into a more typical usage range for solid oral dosage forms, and to lessen the potential for premature disintegration of the tablet during patient administration. Tables 1 and 2 below provide details of the Formulations 1, 2, 3, and 4.

The tablets are manufactured according to wet granulation method known in the art. Differences in the manufacturing process of Formulations 2 and 4 include extragranular incorporation of a portion of microcrystalline cellulose (the tablets of Formulations 1 and 3 incorporated all of this excipient into the tablet granule), as well as the introduction of tablet debossing for all three dose strengths.

TABLE 1
25 mg and 100 mg Formulations

| | Formulation | | | | | | | | | |
|---|---|---|---|---|---|---|---|---|---|---|
| | Formulation 1 | | | | | Formulation 2 | | | | |
| | Common | | | | | Common | | | | |
| Description | Blend | 25 mg | | 100 mg | | Blend | 25 mg | | 100 mg | |
| Component | wt % | mg/tab | | mg/tab | | wt % | mg/tab | | mg/tab | |
| Compound 1 | 32.1 | 25.0 | | 100 | | 31.25 | 25.0 | | 100.0 | |
| Lactose Monohydrate, NF | 32.1 | 25.0 | | 100 | | 30.75 | 24.6 | | 98.4 | |
| Microcrystalline | 24.2 | 18.9 | | 75.5 | | 32.8 | 26.24 | | 105.0 | |
| Cellulose, NF | | | | | | | | | | |
| Povidone, USP | 0.8 | 0.6 | | 2.5 | | 0.80 | 0.64 | | 2.56 | |
| Croscarmellose Sodium, | 9.6 | 7.5 | | 30.0 | | 3.20 | 2.56 | | 10.2 | |
| NF | | | | | | | | | | |
| Colloidal Silicon Dioxide, | 0.6 | 0.5 | | 2.0 | | 0.60 | 0.48 | | 1.92 | |
| NF | | | | | | | | | | |
| Magnesium Stearate, NF | 0.6 | 0.5 | | 2.0 | | 0.60 | 0.48 | | 1.92 | |
| Total | 100.0% | 78.0 | mg | 312.0 | mg | 100.00% | 80.00 | mg | 320.0 | mg |

TABLE 2
300 mg Formulations

| | Formulation | | | | | |
|---|---|---|---|---|---|---|
| | Formulation 3 | | | Formulation 4 | | |
| | Description | | | | | |
| | 300 mg | | | 300 mg | | |
| Component | wt % | mg/tab | | wt % | mg/tab | |
| Compound 1 | 37.5 | 300.0 | | 37.5 | 300 | |
| Lactose Monohydrate, NF | 28.8 | 230.4 | | 24.5 | 196 | |
| Microcrystalline Cellulose, | 22.0 | 176.0 | | 32.8 | 262.4 | |
| NF | | | | | | |
| Povidone, USP | 0.8 | 6.4 | | 0.8 | 6.4 | |
| Croscarmellose Sodium, NF | 9.6 | 76.8 | | 3.2 | 25.6 | |
| Colloidal Silicon Dioxide, NF | 0.7 | 5.6 | | 0.6 | 4.8 | |
| Magnesium Stearate, NF | 0.6 | 4.8 | | 0.6 | 4.8 | |
| Total | 100.0% | 800.0 | mg | 100.0% | 800.0 | mg |

In order to assess the effect of the differences in formulations and manufacturing process on tablet characteristics, dissolution profiles of the formulations are compared. The results provided in Table 3 show percent of tablets dissolved. Table 3 indicate that tablets of Formulations 2 and 4 are fully released and the described differences in the tablets (reduced disintegrant content and associated formulation adjustments, extragranular presence of magnesium stearate, and tablet debossing) did not adversely impact dissolution.

TABLE 3
In Vitro Dissolution Profile Comparison

| Time | Formulations 1 and 3 | | | Formulations 2 and 4 | | |
|---|---|---|---|---|---|---|
| (min) | 25 mg | 100 mg | 300 mg | 25 mg | 100 mg | 100 mg |
| 15 | 78 | 73 | 98 | 110 | 96 | 99 |
| 30 | 88 | 83 | 98 | 104 | 101 | 102 |
| 45 | 92 | 87 | 98 | 103 | 102 | 102 |
| 60 | 94 | 90 | 99 | 103 | 102 | 102 |

### Example 2. Dose-Escalation Study to Determine Pharmacokinetics, Safety and Tolerability of Compound 1 in Subjects with Advanced Malignancies

Compound 1 was evaluated in a dose-escalation study to determine its pharmacokinetics in subjects with advanced malignancies. A total of 52 patients with advanced malignancies were enrolled in 8 cohorts and received Compound 1 doses of 50 mg QD (n=3), 50 mg BID (n=4), 100 mg BID (n=5), 300 mg BID (n=6), 400 mg BID (n=11), 500 mg BID (n=5), 600 mg BID (n=14), and 700 mg BID (n=4). Subjects were provided multiple tablets of 25 mg, 100 mg, or 300 mg of formulations 1 and/or 3 as described in Example 1 to achieve the above indicated doses. Dosing was administered orally with water after at least a 2-hour fast, and the subjects remained fasted for 1 hour after dose administration.

Blood samples for determination of plasma concentrations of Compound 1 were collected at 0, 0.5, 1, 2, 4, 6, 8, and 10 (optional) hours post-dose on Cyclel Day1 and Cyclel Day 15 using lavender top (K2EDTA) Vacutainer® tubes. In addition, blood samples were collected on Cyclel Day8 and on Day1 of each subsequent cycle of treatment for those patients who did not withdraw. Urine samples were not collected for Compound 1 pharmacokinetic analysis in this study.

The plasma samples were assayed by a validated, GLP, LC/MS/MS method with a linear range of 0.020-20.0 μM. Table 4 summarizes the accuracy (Bias %) and precision (CV %) of the assay quality control samples during the analysis of the plasma samples from this study.

TABLE 4
Accuracy and Precision of the Plasma Assay Quality Control Samples

| | --- Low QC --- | | | --- Middle QC --- | | | --- Middle QC --- | | | --- High QC --- | | |
|---|---|---|---|---|---|---|---|---|---|---|---|---|
| Analyte (Unit) | Theo^{a} | Bias % | CV % | Theo^{a} | Bias % | CV % | Theo^{a} | Bias % | CV % | Theo^{a} | Bias % | CV % |
| Compound 1 | 0.060 | −2.9% | 6.6% | 0.80 | −0.6% | 4.5% | 8.0 | 0.0% | 6.5% | 16.0 | −7.2% | 4.5% |
| (μM) | | | | | | | | | | | | |

^{a}Theo = Theoretical or nominal concentration

### Pharmacokinetic Analysis

Standard non-compartmental pharmacokinetic methods were used to analyze the Compound 1 plasma concentration data using Phoenix WinNonlin version 6.0 (Pharsight Corporation, Mountain View, Calif.). Thus, Cₘₐₓ, Cₘᵢₙ and Tₘₐₓ were taken directly from the observed plasma concentration data or imputed in some cases. The terminal-phase disposition rate constant (λ_{z}) was estimated using a log-linear regression of the concentration data in the terminal disposition phase, and t_{1/2} was estimated as ln(2)/λ_{z}. AUC₀₋ₜ and AUC_{0-τ} was estimated using the linear trapezoidal rule for increasing concentrations and the log-trapezoidal rule for decreasing concentrations. At the PK steady-state, the apparent oral-dose clearance, CL/F, was estimated as Dose/AUC_{0-τ}, and Vz/F was estimated as Dose/[AUC_{0-τ}*λ_{z}].

### Statistical Analysis

The log-transformed pharmacokinetic parameters were compared among the BID dose groups using a 1-factor ANOVA with the factor for dose. Dose-dependent exposure parameters (Cₘₐₓ and AUC) were normalized to a common dose before statistical comparisons were made.

The dose-proportionality of Compound 1 steady-state (as observed on Cyclel Day 15) Cₘₐₓ and AUC₀₋₁₂ₕ were evaluated for the BID doses using a power-function regression (e.g., AUC₀₋₁₂ₕ=α·Dose^{β}), where dose proportionality is accepted if 3 is not statistically significantly different from 1.

To determine the food effect on steady-state Compound 1 pharmacokinetics, the log-transformed pharmacokinetic parameters were compared between the treatments using an ANOVA for a 1-way crossover design with the fixed effect for treatment, and random effect for subject. The geometric mean relative bioavailability (reference treatment was the administration in fasted state on Cyclel Day15) and 90% confidence intervals (CI's) for Cₘₐₓ and AUC_{0-τ} were calculated based upon the adjusted means (least square means) from the ANOVA. The food effect on Cₘₐₓ and AUC is considered statistically significant if the 90% CI's exclude the value of 1.

### Results

Results of the study are summarized in the tables below.

TABLE 7
Summary of Compound 1 Steady-State Pharmacokinetic Parameters

| | | Cₘₐₓ | Tₘₐₓ | Cₘᵢₙ | *t_{1/2} | AUC₀₋ₜ | *AUC_{0-τ} | CL/F |
|---|---|---|---|---|---|---|---|---|
| Dose | N | (μM) | (h) | (μM) | (h) | (μM * h) | (μM * h) | (L/h) |
| 50 mg | 3 | 0.396 ± 0.172 | 2.0 | 0.00 ± 0.00 | 2.4 ± 0.26 | 1.39 ± 0.256 | 1.58 ± 0.31 | 73.8 ± 14.7 |
| QD | | | (1.0-4.0) | NC | 2.4 | 1.37 | 1.56 | 73.0 |
| | | 0.365 | | | | | | |
| 50 mg | 4 | 0.742 ± 0.212 | 2.0 | 0.084 ± 0.063 | 2.4 ± 0.56 | 2.74 ± 1.08 | 3.05 ± 1.36 | 43.3 ± 19.2 |
| BID | | | (1.0-3.9) | NC | 2.3 | 2.58 | 2.83 | 40.3 |
| | | 0.715 | | | | | | |
| 100 mg | 5 | 1.23 ± 0.348 | 2.0 | 0.201 ± 0.111 | 3.3 ± 0.75 | 5.32 ± 2.16 | 5.77 ± 2.34 | 45.8 ± 20.9 |
| BID | | 1.19 | (1.0-2.2) | 0.171 | 3.2 | 4.97 | 5.38 | 42.4 |
| 300 mg | 5 | 2.48 ± 0.515 | 2.0 | 0.287 ± 0.146 | 3.9 ± 2.1 | 8.92 ± 0.841 | 9.78 ± 0.86 | 70.4 ± 6.19 |
| BID | | 2.44 | (1.0-2.0) | 0.251 | 3.5 | 8.88 | 9.75 | 70.2 |
| 400 mg | 8 | 4.39 ± 2.02 | 2.0 | 0.624 ± 0.339 | 2.7 ± 0.62 | 16.7 ± 6.79 | 19.6 ± 7.43 | 62.3 ± 52.3 |
| BID | | 3.88 | (1.0-6.0) | 0.523 | 2.6 | 15.0 | 17.6 | 51.8 |
| 500 mg | 5 | 4.82 ± 2.26 | 2.0 | 0.604 ± 0.260 | 2.4 ± 0.37 | 18.2 ± 6.46 | 20.6 ± 6.82 | 60.5 ± 19.1 |
| BID | | 4.48 | (2.0-2.4) | 0.562 | 2.4 | 17.3 | 19.7 | 58.0 |
| 600 mg | 12 | 4.82 ± 2.16 | 2.0 | 0.932 ± 0.704 | 3.3 ± 0.97 | 19.5 ± 8.4 | 22.9 ± 10.0 | 66.4 ± 18.6 |
| BID | | 4.52 | (1.0-2.1) | 0.731 | 3.2 | 18.3 | 21.6 | 63.5 |
| 700 mg | 4 | 6.23 ± 2.09 | 3.0 | 1.32 ± 0.417 | 3.0 ± 1.2 | 30.8 ± 10.5 | 35.8 ± 15.5 | 49.5 ± 17.1 |
| BID | | 6.00 | (2.0-4.5) | 1.26 | 2.9 | 29.6 | 33.9 | 47.2 |

P-Values from a 1-Factor ANOVA (Factor = Dose) of Log-Transformed Exposures after Dose Normalization

| | | |
|---|---|---|
| Dose | 0.0051 | 0.0961 |

Values are mean ± SD and geometric mean except that Tₘₐₓ is reported as median (range)
*t_{1/2} and hence AUC_{0-12 h} values could not be estimated for 4 subjects.

Administered in the fasted state, Compound 1 peak plasma concentrations (Cₘₐₓ) were typically observed at 2 hours (median Tₘₐₓ) post-dose, and subsequently, Compound 1 plasma concentrations declined in a mono- or bi-exponential fashion. The terminal phase t_{1/2} appeared to be dose independent with a geometric mean value of 2.9 hours for all the subjects (n=42, inter-subject CV=35.2%) who had estimable t_{1/2} values on Cyclel Day15.

Following repeat BID dosing of Compound 1, the steady-state of PK was observed on or before Day 8 of dosing, as judged by the time course of trough plasma concentrations. The trough concentrations for 50 mg QD dose were low and generally not quantifiable (>BQL). The relatively short t_{1/2} of Compound 1 suggests that the PK steady-state should be reached within 2 days of dosing.

For the 7 BID doses, the average drug accumulation index, or the geometric mean ratio (GMR) of Cₘₐₓ and AUC_{0-τ} on Day 15 vs. Day1, was 1.16 and 1.33, respectively, which is significantly greater than the extent of accumulation implied by the t_{1/2} value of 2.9 hours which in turn implies enterohepatic recycling or biliary recycling. There was no evidence of systemic accumulation following repeat 50 mg QD administration.

Compound 1 exposures were slightly less than proportional to dose. For the BID doses at the steady state, the power-function regression analysis produced dose-proportionality equation for Cₘₐₓ=0.330·Dose^{0.779} (p=0.0025 for β=1) and AUC₀₋₁₂ₕ=0.103·Dose^{0.843} (p=0.043 for β=1). The 90% CI of the exponent, β, of the power function (or equivalently the slope of the log-transformed equation) was (0.664, 0.895) for Cₘₐₓ and (0.717, 0.969) for AUC₀₋₁₂ₕ. Since the upper bounds of 90% CI's of j were less than 1, Compound 1 exposures (Cₘₐₓ and AUC₀₋₁₂ₕ) were statistically significantly deviated from proportionality to dose over the range of 50 to 700 mg BID. The degree of sub-linearity for dose proportionality was moderate as indicated by the 3 point estimate of 0.843 for AUC (e.g., the equation estimates ˜7-fold increase in AUC with a 10-fold increase in dose).

Compound 1 plasma exposures exhibited a moderate inter-subject variability at the steady-state, with the coefficient of variability (CV %) ranging from 20.8% to 46.8% for Cₘₐₓ, and from 8.8 to 44.5% for AUC₀₋₁₂ₕ, respectively.

In an expanded cohort, the food effect of a standardized high-fat meal on Compound 1 steady-state pharmacokinetics was evaluated for the 600 mg BID dose. The results are summarized in Table 8 below.

TABLE 8
The Effect of a High-Fat Meal on Compound 1 PK

| | | Cₘₐₓ | Tmax | *AUC_{0-12 h} |
|---|---|---|---|---|
| Treatment | N | (μM) | (h) | (μM * h) |
| Fasted | 12 | 4.82 ± 2.16 | 2.0 | 22.9 ± 9.99 |
| | | 4.52 | (1.0-2.1) | 21.6 |
| Fed | 9 | 4.53 ± 2.52 | 6.0 | 29.5 ± 18.2 |
| | | 4.03 | (2.0-8.0) | 25.8 |

P-Values from a 1-Way Crossover ANOVA (Reference = Fasted)
of Log-Transformed Exposures

| | | |
|---|---|---|
| 9 | 0.561 | 0.174 |

Geometric Mean Ratios and 90% Confidence Intervals
(Reference = Fasted)**

| | | | |
|---|---|---|---|
| Fed | 9 | 0.897 (0.645-1.25) | 1.22 (0.952-1.57) |

Values are mean ± SD and geometric mean except that Tₘₐₓ is reported as median (range)
*Since t½ values could not be estimated in majority of subjects with dosing in the fed state, concentration values at 12 h post dose were imputed from the pre-dose trough on the morning of Cycle 2_Day 1.
**Statistical analysis performed using the logarithmically transformed drug exposure data for the 9 subjects who completed the food effect study.

Administration of the high-fat meal prolonged the mean Compound 1 Tₘₐₓ by 4 hours, decreased the geometric mean Cₘₐₓ by approximately 10% and increased the geometric mean AUC₀₋₁₂ₕ by 22%. The 90% CI's of the GMR point estimates for Cₘₐₓ and AUC₀₋₁₂ₕ spanned the value of 1, and the corresponding p values from the 1-way crossover ANOVA were greater than 0.05, indicating the effect on Compound 1 plasma exposures from a high-fat meal was not statistically significant. The magnitude of the food effect on Compound 1 exposures also does not appear to be clinically important. Although the effect of a medium-fat meal was not studied, it is expected that the change in Compound 1 PK will be even less pronounced compared to that with a high-fat meal.

### Oral Bioavailability and Systemic Clearance

The oral bioavailability (F) and systemic clearance (CL) were estimated. Pooling the data from 42 subjects who had evaluable oral-dose clearance (CLₒᵣₐₗ=CL/F) on Cyclel Day15, the geometric mean value was 55.3 L/h (range: 23.3-180 L/h; inter-subject CV %=44.3%). The value of F and CL for Compound 1 may be estimated using the equations of F=QH/(QH+CL/F), and CL=(QH*CL/F)/(QH+CL/F) (see Gibaldi M and Perrier D, Pharmacokinetics, 2^{nd} Ed., Informa Healthcare USA, New York 2007), where Q is the typical value of human hepatic blood flow rate (approximately 87 L/h). This method of estimation assumes near complete oral absorption and the liver being the primary organ for drug clearance. Since the observed renal excretion of unchanged Compound 1 was less than 3% of IV dose in the mice, monkeys and dogs, the assumption that Compound 1 is almost entirely cleared by the liver seems to be a reasonable approximation. However, sub-linear dose-exposure relationship suggests that the fraction of oral dose absorbed (Fa) decreases with increasing Compound 1 dose. Based on the data from preclinical PK studies (not shown), Fa may be estimated as 48% and 81%, in the cynomolgus monkeys and beagle dogs, respectively. Therefore, the human Fa of Compound 1 is estimated to be 64% (the mean value in the cynomolgus monkey and beagle dog). The above equation was modified to incorporate the term of Fa to accommodate an incomplete absorption: CL=(QH*Fa*CL/F)/(QH+Fa*CL/F). Using the mean estimates of Fa=64%, QH=87 L/h and CL/F=55.3 L/h, the mean systemic clearance, CL, is estimated to be 25 L/h, and the mean absolute bioavailability (F) is estimated to be 45% (CL/CLₒᵣₐₗ). Since the estimated hepatic extraction ratio is 29% (CL/QH), Compound 1 can be considered as a low clearance compound. Expressed in terms of percent hepatic blood flow, the estimated systemic clearance in human (29%) is comparable to that observed in the cynomolgus monkey (31%) and beagle dog (26%).

The unbound fraction of Compound 1 in plasma was determined to be 3.1%, and the highest steady-state mean unbound daily AUC₀₋₂₄ₕ (=2×AUC₀₋₁₂ₕ) was calculated to be 2.2 μM*h, associated with the 700 mg BID dose. This value was well below the NOAEL unbound AUC₀₋₂₄ₕ Of 7.9 μM*h observed in the male dogs in the 500 mg/kg/day dose group in the 28-day GLP toxicology study.

### SUMMARY

In summary, following oral dose administration in the fasted state, the peak plasma concentration of Compound 1 was typically attained at 2 hours post-dose. Compound 1 was eliminated with a geometric mean terminal disposition half-life of 2.9 hours. Systemic accumulation following BID dosing increased mean Compound 1 Cₘₐₓ and AUC_{0-τ} by 16% and 33%, respectively, suggesting an “effective” half-life of 4-6 hours. Increases in Compound 1 Cₘₐₓ and AUC_{0-τ} were less than proportional to dose. The slightly lower than dose proportional relationship was most likely due to limited rate and/or extent of intestinal absorption for this compound at higher doses. A high-fat meal delayed Compound 1 median Tₘₐₓ by 4 hours but did not cause clinically significant change in Compound 1 plasma exposures. Therefore, Compound 1 may be dosed without regard to food. Moderate inter-subject variability was observed for Compound 1 plasma exposure at the steady-state following administration in the fasted state. The highest steady-state mean unbound 0-24 hour AUC (2.2 μM*h) observed in this study (700 mg BID dose group) was well below the NOAEL unbound AUC₀₋₂₄ₕ of 7.9 μM*h observed in the 28-day GLP toxicology study.

### Example 3. Compound 1 in Combination with MK-3475

Compound 1 was evaluated in a study to determine its pharmacokinetics in subjects with various cancers. The subjects were not limited to a specific cancers and study include subjects with various cancers. Phase 1 was the dose-escalation phase, which included cohorts of subjects treated with Compound 1 at initial doses of 25 mg BID, 50 mg BID, and 100 mg BID in combination with MK-3475 (also known as pembrolizumab, lambrolizumab, and Keytruda®) at 2 mg/kg every 3 weeks (Q3W), and Compound 1 at 300 mg BID in combination with MK-3475 at 200 mg/kg Q3W. One treatment cycle consisted of 21 days. A minimum of 3 subjects were enrolled and treated in each cohort, and all 3 subjects were observed for a minimum of 42 days (6 weeks) before the subsequent cohort began enrollment. Subjects must have received the cohort-specific dose of Compound 1 for at least 80% of the doses during the 42-day dose-limiting toxicity (DLT) observation period, and must have received 2 doses of MK-3475 during that 42-day period, or must have experienced a DLT to be included in the cohort review for DLTs. Additional subjects were enrolled in a cohort to achieve the minimum of 3 evaluable subjects if dropouts or dose interruptions or reductions occur that result in a subject being non-evaluable for DLTs. When the preliminary safety of 50 mg BID and 100 mg BID was established, additional subjects with melanoma were enrolled at 50 mg BID for a total of 9 subjects. An additional safety cohort was opened at 100 mg BID in parallel to 300 mg BID being tested. This may also be limited to subjects with melanoma, NSCLC, or specific cancer types from among those included in Phase 1. The RP2D was selected from the evaluated safety expansions. All subjects in these safety expansions were treated with MK-3475 200 mg Q3W.

Compound 1 was self-administered orally BID and continued BID during the 21-day cycle for an every-3-week dose schedule of MK-3475. The maximum tolerated dose (MTD) of Compound 1 (or population adjusted dose (PAD)) defined during Phase 1 was used for Phase 2. All BID doses were taken morning and evening, approximately 12 hours apart without respect to food. If a dose was missed by more than 4 hours, that dose was skipped and was resumed at the scheduled time.

Subjects arrived at clinic having withheld their morning dose of Compound 1. Pharmacokinetic samples were obtained at the visits of Cycle 1 Day 1, Cycle 1 Day 8 and Cycle 2 Day 1. After the pre-dose (which defined as within 24 hours before administration of MK-3475 and before administration of Compound 1), PK sample was drawn, subjects would take Compound 1 and then begin infusion of MK-3475. The exact date and time of the PK blood draws were recorded in the eCRF along with the date and time of the last dose of study drug and details of the last meal preceding the blood draw.

The plasma samples were assayed by a validated, GLP, LC/MS/MS method with a linear range of 0.020 to 20 μM and a limit of quantification of 0.020 μM.

The planned PK time points were used for preliminary PK analyses. Due to limited PK sampling up to 6 or 8 hours post-dose, a C12 h values for the PK visit of C1D10 were imputed from the pre-dose concentration on the same day in order to calculate AUC₀₋₁₂ₕ. Standard non-compartmental analysis (NCA) methods were used to analyze Compound 1 plasma concentration data using Phoenix WinNonLin version 6.4 (Pharsight Corporation, Mountain View, Calif.).

### Pharmacokinetic Model

In non-compartmental analysis (NCA), EPA showed approximate dose-proportional exposures, indicating a constant rate of clearance independent of EPA concentration. (Kleiber M., J Theor Biol. 1975; 53(1): 199-204). For the base structural model development, standard compartmental PK models comprising of the first-order kinetics of oral absorption, 1, 2, or 3-compartment distribution, and linear elimination from the central compartment were tested for their ability to characterize the observed plasma concentration-time profiles of EPA.

After a final base structural model was identified, the effect of covariates including body weight (BW), age and gender on the PK parameters was first explored using visual inspection for correlations between the random variables (q) of a parameter (e.g., CL/F) and the covariate. A covariate that showed a tentative correlation was then incorporated in the model. A covariate contributing at least a 6.63 reduction in the objective function (α=0.01) were considered significant in forward selection process, and a covariate was considered significant if it contributed at least a 10.8 increase in the objective function value (α=0.001) when removed from the model in backward elimination process. After the stepwise selection procedure was complete, the model was also checked for possible simplifications of covariate equations, such as power functions that could be reduced to linear functions (power term approximately 1.0) if justified from theoretical consideration.

After completing the model development process, the final model was assessed for its predictive performance by two methods of validation: visual predictive check (VPC) and internal validation. A total of 1000 replications of the analysis datasets were simulated using the final model for VPC. Statistics of interest (50th [median], 10-90th and 5-95th percentiles) were calculated from the simulated concentration values at each simulated sampling time point. Graphical model evaluation results were prepared, including an overlay of the original data on the prediction intervals based on the simulated replicate datasets. As internal validation, the final model was tested on a subset of data (in this case, the PK data from the first dose on Day 1). A lack of significant change in the parameter values estimated supports the model's capability to fit the data observed.

### Pharmacodynamic Model

A mechanistic population PD model was constructed to capture the principal components of bioconversion of TRP to KYN catalyzed by IDO1 and TPO in parallel. In this model the plasma concentration of KYN is the dependent variable (DV). TRP, one of the essential amino acids, is an abundant endogenous chemical in human with an average plasma concentration observed at ˜60 μM in this study. In comparison, KYN, one of the catabolic products of TRP, is produced in a relatively small quantity (2-3% of TRP). With the expected homeostasis maintained for TRP, an inhibition of KYN production is not expected to alter significantly the level of TRP. Therefore, this PD model did not include the rate of formation for TRP; the concentrations of TRP at sampled time points were observed values and used as model inputs. It is assumed that the inhibition of IDO1 by EPA follows a sigmoidal Iₘₐₓ/IC₅₀ model:

$$I = Imax \times \frac{IC {50}^{n}}{IC {50}^{n} + {[ EPA ]}^{n}}$$

where [EPA] is EPA plasma concentration, IC₅₀ is the [EPA] that causes 50% of maximal inhibition, Iₘₐₓ, which is assumed to be 100% in this model (as almost complete inhibition of IDO1 was observed at high concentrations of EPA in vitro), and n is the Hill factor. The bioconversion from TRP to KYN by parallel pathways via IDO1 and TPO is described by the following equation:

$$\frac{d [ KYN ]}{dt} = [ TRP ] \times ( k 1 - I * k 1 + k 2 ) - [ KYN ] \times kdeg$$

where [TRP] and [KYN] are the plasma concentrations of TRP and KYN respectively, k1 and k2 are the KYN formation rate constants via IDO1 and TPO respectively, and kdeg is the rate constant of KYN degradation. Estimates of the initial values of [KYN] were provided by:

$$[ KYN ] = [ TRP ] \times \frac{( k 1 + k 2 )}{kdeg}$$

The procedures of model building and covariate testing were similar to those described above for the PK model. The primary endpoint of this PD model was to the estimated value of IC₅₀.

The results of the study are shown below.

TABLE 9
Compound 1 First Dose PK Parameters (by Cohort and Dose)

| | Dose | | Cₘₐₓ | tₘₐₓ | AUC₀₋ₜ |
|---|---|---|---|---|---|
| Cohort | (mg) | n | (μM) | (h) | (μM * h) |
| 1 | 25 | 3 | 0.231 ± 0.151 | 2.00 | 0.711 ± 0.349 |
| | | | (0.199) | (1.00, 2.00) | (0.650) |
| 2 | 50 | 8 | 0.574 ± 0.216 | 2.00 | 1.60 ± 0.507 |
| | | | (0.543) | (0.50, 4.00) | (1.53) |
| 3 | 100 | 4 | 0.632 ± 0.508 | 4.00 | 2.09 ± 1.15 |
| | | | (0.521) | (1.00, 4.00) | (1.89) |
| 4 | 50 | 12 | 0.512 ± 0.224 | 1.00 | 1.32 ± 0.698 |
| (MEL) | | | (0.474) | (0.50, 2.00) | (1.20) |
| 5 | 100 | 14 | 0.852 ± 0.340 | 1.50 | 2.58 ± 0.778 |
| | | | (0.787) | (1.00, 4.00) | (2.46) |
| 6 | 300 | 7* | 2.22 ± 1.72 | 2.00 | 7.47 ± 3.32 |
| | | | (1.79) | (1.00, 4.00) | (6.92) |
| 7 | 300 | 12** | 2.33 ± 0.925 | 2.00 | 7.38 ± 2.86 |
| | | | (2.17) | (0.50, 6.00) | (6.88) |

*Subject 103006 was excluded from PK analysis due to incomplete PK
profile (only three post-dose PK samples were collected at 0.5, 1
and 2 hours).
**Subject 101025 was actually a Part 2 subject (NSCLC PD-L1 High)
that was mistakenly categorized as a Part 1 Cohort 7 subject in the
previous PK Update (June 2016).
MEL: melanoma

| | | Cₘₐₓ | tₘₐₓ | AUC₀₋ₜ |
|---|---|---|---|---|
| Dose (mg) | n | (μM) | (h) | (μM * h) |
| 25 | 3 | 0.231 ± 0.151 | 2.00 | 0.711 ± 0.349 |
| | | (0.199) | (1.00, 2.00) | (0.650) |
| 50 | 20 | 0.537 ± 0.217 | 2.00 | 1.43 ± 0.630 |
| | | (0.500) | (0.50, 4.00) | (1.33) |
| 100 | 18 | 0.803 ± 0.378 | 2.00 | 2.47 ± 0.861 |
| | | (0.719) | (1.00, 4.00) | (2.32) |
| 300 | 19* | 2.29 ± 1.23 | 2.00 | 7.41 ± 2.95 |
| | | (2.02) | (0.50, 6.00) | (6.89) |

Values are presented in the format of “Mean ± SD (Geometric
Mean) except that “Median (Min, Max)” for Tₘₐₓ
*Subject 103006 (Cohort 6) was excluded from PK analysis due to
incomplete PK profile (only three post-dose PK samples were
collected at 0.5, 1 and 2 hours);
*Subject 101025 was actually a Part 2 subject (NSCLC PD-L1 High)
that was mistakenly categorized as a Part 1 Cohort 7 subject in
previous PK Update.

TABLE 10
Compound 1 Steady State (C1D8) PK Parameters (by Cohort and Dose)

| | Dose | | Cₘₐₓ | tₘₐₓ | Cₘᵢₙ | AUC_{0-12 h} | CL/F |
|---|---|---|---|---|---|---|---|
| Cohort | (BID) | n | (μM) | (h) | (μM) | (μM * h) | (L/h) |
| 1 | 25 | 4 | 0.307 ± 0.150 | 1.00 | 0.0423 ± 0.013 | 1.27 ± 0.230 | 46.2 ± 8.91 |
| | | | | (1.00, | | (1.25) | (45.6) |
| | | | (0.276) | 2.00) | (0.0407) | | |
| 2 | 50 | 7* | 0.603 ± 0.227 | 2.00 | 0.0583 ± 0.037 (NC) | 2.53 ± 1.02 | 52.6 ± 22.4 |
| | | | | (1.00, | | (2.34) | (48.7) |
| | | | (0.550) | 4.00) | | | |
| 3 | 100 | 4 | 0.956 ± 0.497 | 2.00 | 0.0983 ± 0.0601 | 3.91 ± 1.35 | 67.4 ± 34.9 |
| | | | | (1.00, | | (3.67) | (62.2) |
| | | | (0.814) | 2.00) | (0.0856) | | |
| 4 | 50 | 12 | 0.442 ± 0.232 | 1.50 | 0.0413 ± 0.0363 (NC) | 1.77 ± 1.12 | 78.3 ± 28.9 |
| (MEL) | | | | (1.00, | | (1.58) | (72.3) |
| | | | (0.403) | 4.00) | | | |
| 5 | 100 | 12 | 0.905 ± 0.421 | 2.00 | 0.097 ± 0.0774 (NC) | 3.79 ± 1.43 | 69.6 ± 28.4 |
| | | | | (1.00, | | (3.53) | (64.6) |
| | | | (0.803) | 4.00) | | | |
| 6 | 300 | 7 | 2.71 ± 1.22 | 2.00 | 0.285 ± 0.127 | 12.3 ± 6.09 | 64.8 ± 23.2 |
| | | | (2.50) | (0.50, | (0.264) | (11.3) | (60.6) |
| | | | | 4.00) | | | |
| 7 | 300 | 12** | 2.75 ± 1.22 | 2.00 | 0.276 ± 0.205 | 12.2 ± 5.92 | 65 ± 23.4 |
| | | | (2.52) | (1.00, | (0.223) | (11.3) | (60.7) |
| | | | | 4.00) | | | |

*Subject 101009 was excluded from PK analysis due to lack of the pre-dose PK sample on C1D8;
**Subject 101025 was actually a Part 2 subject (NSCLC PD-L1 High) that was mistakenly categorized
as a Part 1 Cohort 7 subject in previous PK Update.

| Dose | | Cₘₐₓ | tₘₐₓ | Cₘᵢₙ | AUC_{0-12 h} | CL/F |
|---|---|---|---|---|---|---|
| (BID) | n | (μM) | (h) | (μM) | (μM * h) | (L/h) |
| 25 | 4 | 0.307 ± 0.150 | 1.00 | 0.0423 ± 0.013 | 1.27 ± 0.230 | 46.2 ± 8.91 |
| | | (0.276) | (1.00, | (0.0407) | (1.25) | (45.6) |
| | | | 2.00) | | | |
| 50 | 19* | 0.502 ± 0.237 | 2.00 | 0.0476 ± 0.0365 | 2.05 ± 1.12 | 68.8 ± 29.0 |
| | | (0.452) | (1.00, | (NC) | (1.83) | (62.5) |
| | | | 4.00) | | | |
| 100 | 16 | 0.917 ± 0.424 | 2.00 | 0.0973 ± 0.0715 | 3.82 ± 1.37 | 69.1 ± 28.9 |
| | | (0.806) | (1.00, | (NC) | (3.57) | (64.0) |
| | | | 4.00) | | | |
| 300 | 19** | 2.74 ± 1.19 | 2.00 | 0.279 ± 0.177 | 12.3 ± 5.81 | 64.9 ± 22.7 |
| | | (2.51) | (0.50, | (0.237) | (11.3) | (60.7) |
| | | | 4.00) | | | |

Subjects who experienced dose reduction before C4D1

| | | | | | | |
|---|---|---|---|---|---|---|
| 50 | 3 | 0.557 ± 0.260 | 2.00 | 0.0700 ± 0.0203 | 2.41 ± 0.980 | 52.0 ± 17.1 |
| | | (0.519) | (2.00, | (0.0682) | (2.29) | (49.8) |
| | | | 2.00) | | | |
| 300 | 4 | 3.18 ± 1.28 | 2.00 | 0.364 ± 0.126 | 14.6 ± 7.40 | 55.4 ± 23.0 |
| | | (3.00) | (0.50, | (0.346) | (13.3) | (51.3) |
| | | | 4.00) | | | |

Values are presented in the format of “Mean ± SD (Geometric Mean) except that “Median (Min, Max)”
for Tₘₐₓ
NC: not calculable due to at least one PK sample was BQL;
*Subject 101009 (Cohort 2) was excluded from PK analysis due to lack of the pre-dose PK sample on
C1D8;
**Subject 101025 was actually a Part 2 subject (NSCLC PD-L1 High) that was mistakenly categorized
as a Part 1 Cohort 7 subject in previous PK Update.
Subjects experiencing dose reduction by C4D1:
50 mg BID: 102006 (Cohort 2, 50 mg BID + 2 mg/mg Q3W), 102012 (Cohort 4, 50 mg BID + 200 mg
Q3W), 102019 (Cohort 4, 50 mg BID ± 200 mg Q3W);
300 mg BID: 101015 (Cohort 6, 300 mg BID + 200 mg Q3W), 103006 (Cohort 6, 300 mg
BID + 200 mg
Q3W), 104006 (Cohort 6, 300 mg BID + 200 mg Q3W), and 101022 (Cohort 7, 300 mg BID + 200 mg
Q3W expansion)

TABLE 11
Projected Steady State IDO1 Inhibitions on C1D8
(by Cohort and Dose)

| Cohort | Dose (BID) | n | Imax (%) | Imin (%) | Iavg (%) |
|---|---|---|---|---|---|
| 1 | 25 | 4 | 79 ± 9.4 | 37 ± 7.3 | 54 ± 4.9 |
| | | | (78) | (36) | (54) |
| 2 | 50 | 7 | 88 ± 6.7 | 41 ± 20 | 65 ± 11 |
| | | | (88) | (NC) | (64) |
| 3 | 100 | 4 | 91 ± 7.2 | 55 ± 15 | 73 ± 7.5 |
| | | | (90) | (53) | (73) |
| 4 | 50 | 12 | 85 ± 4.9 | 31 ± 22 | 54 ± 12 |
| (MEL) | | | (84) | (NC) | (53) |
| 5 | 100 | 12 | 91 ± 4.8 | 48 ± 26 | 71 ± 9.4 |
| | | | (91) | (NC) | (71) |
| 6 | 300 | 7 | 97 ± 1.1 | 78 ± 6.5 | 89 ± 3.6 |
| | | | (97) | (78) | (88) |
| 7 | 300 | 12 | 97 ± 1.3 | 74 ± 12 | 87 ± 5.5 |
| | | | (97) | (73) | (87) |

| Dose (BID) | n | Imax (%) | Imin (%) | Iavg (%) |
|---|---|---|---|---|
| 25 | 4 | 79 ± 9.4 | 37 ± 7.3 | 54 ± 4.9 |
| | | (78) | (36) | (54) |
| 50 | 19 | 86 ± 5.7 | 35 ± 21 | 58 ± 13 |
| | | (86) | (NC) | (57) |
| 100 | 16 | 91 ± 5.2 | 50 ± 23 | 72 ± 8.8 |
| | | (91) | (NC) | (71) |
| 300 | 19 | 97 ± 1.2 | 76 ± 10 | 88 ± 4.8 |
| | | (97) | (75) | (88) |

Values are presented as “Mean ± SD (Geometric Mean);
Projected PD (IDO) inhibition was calculated as Conc/(Conc + EC50) * 100 (%) in which EC50 = 70 nM;
NC: not calculable due to at least one PK sample was BQL (thus the PD inhibition was projected as 0%);
Time-averaged IDO1 inhibition was calculated using the linear-up-log-down method;

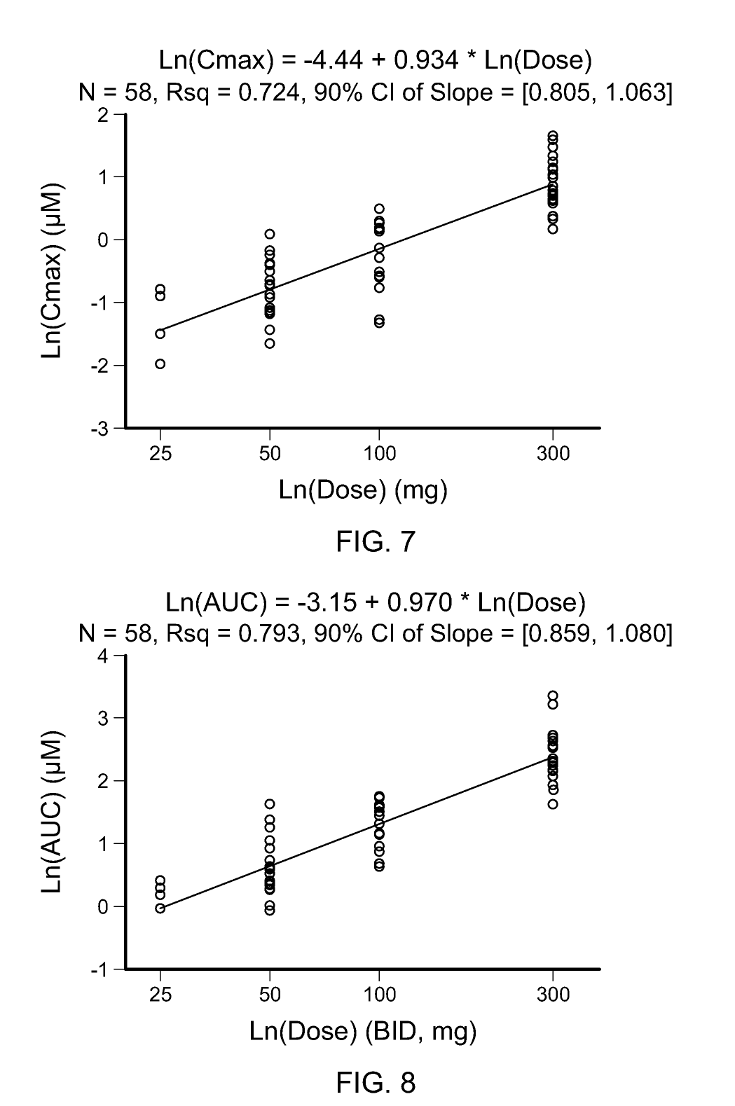

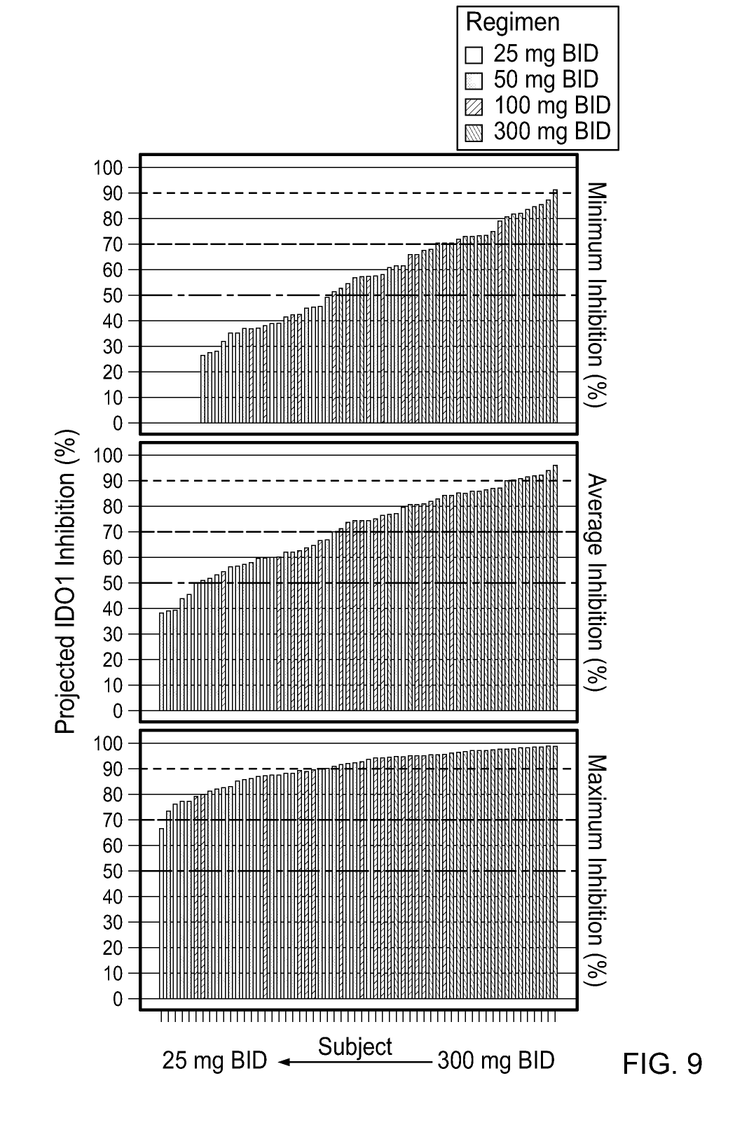

The results are also shown in the figures. FIG. 4 and FIG. 5 are graphs of Compound 1 plasma concentrations (Mean±SE) by dose following the first dose (FIG. 4) and at steady state (FIG. 5). FIG. 6 is a graph of Compound 1 plasma concentrations (Mean±SE) on C1D8 and C2D1. FIG. 7 and FIG. 8 are graphs of the dose proportional PK of Compound 1 on C1D8 (all cohorts in part 1). FIG. 9 shows the waterfall plots of projected percent IDO1 inhibition for various doses (N=58).

TABLE 12
First dose pharmacokinetic parameters

| | | Cₘₐₓ | tₘₐₓ | AUC₀₋ₜ |
|---|---|---|---|---|
| Diagnosis | n | (μM) | (h) | (μM * h) |
| DLBCL | 6 | 0.732 ± 0.370 | 3.00 | 2.29 ± 1.16 |
| | | (0.664) | (1.00, 4.00) | (1.90) |
| GU | 19 | 0.986 ± 0.492 | 2.00 | 2.96 ± 1.28 |
| | | (0.878) | (1.00, 6.00) | (2.71) |
| MEL | 9 | 0.870 ± 0.347 | 2.00 | 2.48 ± 0.773 |
| | | (0.809) | (1.00, 4.00) | (2.37) |
| NSCLC PD-L1 | 6 | 1.02 ± 0.573 | 1.50 | 2.88 ± 1.30 |
| High | | (0.878) | (1.00, 4.00) | (2.62) |
| NSCLC PD-L1 | 9 | 0.914 ± 0.343 | 1.00 | 2.77 ± 0.839 |
| Low/NE | | (0.857) | (0.50, 6.00) | (2.66) |
| OC | 32 | 0.967 ± 0.405 | 2.00 | 3.16 ± 1.10 |
| | | (0.886) | (1.00, 4.00) | (2.99) |
| RCC | 2 | 1.35, 0.418 | 2.0, 3.0 | 3.58, 1.74 |
| SCCHN | 19 | 0.769 ± 0.439 | 2.00 | 2.32 ± 1.10 |
| | | (0.649) | (1.00, 6.00) | (2.06) |
| TNBC | 34 | 1.03 ± 0.574 | 2.00 | 3.08 ± 1.39 |
| | | (0.898) | (0.50, 6.00) | (2.82) |
| All | 136 | 0.937 ± 0.469 | 2.00 | 2.87 ± 1.19 |
| | | (0.830) | (0.50, 6.00) | (2.63) |

Values are presented in the format of “Mean ± SD (Geometric Mean) except that “Median (Min, Max)” for Tₘₐₓ.
DLBCL: diffuse large B-cell lymphoma;
GU: genitourinary cancer;
MEL: melanoma;
NSCLC: non-small-cell lung carcinoma;
OC: ovarian cancer;
RCC: renal cell cancer;
SCCHN: squamous cell carcinoma of the head and neck;
TNBC: triple negative breast cancer.

TABLE 13
Steady State (C1D8) Pharmacokinetic Parameters

| | | Cₘₐₓ | tₘₐₓ | Cₘᵢₙ | AUC_{0-12 h} | CL/F |
|---|---|---|---|---|---|---|
| Diagnosis | n | (μM) | (h) | (μM) | (μM * h) | (L/h) |
| DLBCL | 2 | 0.883, 0.904 | 4.0, 4.0 | 0.12, 0.08 | 5.32, 3.99 | 21.4, 28.6 |
| GU | 16 | 1.10 ± 0.400 | 2.00 | 0.154 ± 0.203 | 4.98 ± 3.01 | 28.1 ± 10.0 |
| | | (1.03) | (0.50, | (NC) | (4.41) | (25.9) |
| | | | 4.00) | | | |
| MEL | 9 | 1.00 ± 0.347 | 2.00 | 0.0956 ± 0.0409 | 3.68 ± 0.852 | 33.0 ± 10.0 |
| | | (0.945) | (1.00, | | | (31.9) |
| | | | 4.00) | (0.0882) | (3.57) | |
| NSCLC PD- | 6 | 0.939 ± 0.549 | 3.00 | 0.159 ± 0.0777 | 4.43 ± 1.81 | 29.2 ± 10.4 |
| L1 High | | (0.815) | (0.50, | | (4.15) | (27.5) |
| | | | 6.00) | (0.145) | | |
| NSCLC PD- | 5 | 0.879 ± 0.156 | 2.00 | 0.125 ± 0.0468 | 4.35 ± 0.828 (4.3) | 26.9 ± 4.57 |
| L1 Low/NE | | (0.867) | (1.00, | | | (26.6) |
| | | | 4.00) | (0.116) | | |
| OC | 28 | 1.20 ± 0.487 | 2.00 | 0.0937 ± 0.0742 (NC) | 4.66 ± 2.11 | 27.9 ± 8.82 |
| | | (1.11) | (1.00, | | (4.34) | (26.3) |
| | | | 6.00) | | | |
| SCCHN | 17 | 0.987 ± 0.612 | 2.00 | 0.108 ± 0.0784 | 4.29 ± 2.30 | 33.3 ± 15.5 |
| | | (0.821) | (0.50, | | (3.81) | (29.9) |
| | | | 6.00) | (0.0893) | | |
| TNBC | 24 | 1.21 ± 0.433 | 2.00 | 0.117 ± 0.0712 | 5.01 ± 2.09 | 25.7 ± 8.22 |
| | | (1.15) | (0.50, | | (4.69) | (24.3) |
| | | | 4.00) | (0.100) | | |
| All | 107 | 1.10 ± 0.467 | 2.00 | 0.116 ± 0.102 | 4.62 ± 2.14 | 28.7 ± 10.3 |
| | | (1.01) | (0.50, | (NC) | (4.26) | (26.8) |
| | | | 6.00) | | | |
| Dose | 7 | 1.04 ± 0.551 | 2.00 | 0.110 ± 0.0549 | 5.10 ± 3.59 | 28.4 ± 11.1 |
| Reduction | | (0.953) | (1.00, | | (4.42) | (25.8) |
| By C4D1 | | | 4.00) | (0.0978) | | |

Values are presented in the format of “Mean ± SD (Geometric Mean) except that “Median (Min, Max)” for Tmax.
NC: not calculable due to at least one PK sample was BQL.
Subjects experiencing dose reduction by C4D1: GU: 107014;
NSCLC PD-L1 High: 101025;
OC: 102042, 109010, 113002 and 116003;
SCCHN: 101045.
DLBCL: diffuse large B-cell lymphoma;
GU: genitourinary cancer;
MEL: melanoma;
NSCLC: non-small-cell lung carcinoma;
OC: ovarian cancer;
RCC: renal cell cancer;
SCCHN: squamous cell carcinoma of the head and neck;
TNBC: triple negative breast cancer.

TABLE 14
Projected Steady State IDO Inhibitions on C1D8

| Diagnosis | Dose (BID) | N* | Imax (%) | Imin (%) | Iavg (%) |
|---|---|---|---|---|---|
| DLBCL | 100 mg | 2 | 93, 93 | 63, 53 | 81, 74 |
| GU | 100 mg | 16 | 93 ± 2.5 | 55 ± 22 | 75 ± 9.3 |
| | | | (93) | (NC) | (75) |
| MEL | 100 mg | 9 | 93 ± 2.4 | 55 ± 10 | 73 ± 5.8 |
| | | | (93) | (55) | (73) |
| NSCLC PD- | 100 mg | 6 | 91 ± 4.3 | 67 ± 9.2 | 79 ± 5.2 |
| L1 High | | | (91) | (67) | (79) |
| NSCLC PD- | 100 mg | 5 | 92 ± 1.2 | 62 ± 11 | 78 ± 4.8 |
| L1 Low/NE | | | (92) | (61) | (78) |
| OC | 100 mg | 28 | 94 ± 2.8 | 52 ± 21 | 74 ± 8.7 |
| | | | (94) | (NC) | (74) |
| SCCHN | 100 mg | 17 | 91 ± 6.7 | 55 ± 14 | 74 ± 8.6 |
| | | | (90) | (54) | (73) |
| TNBC | 100 mg | 24 | 94 ± 1.8 | 58 ± 12 | 76 ± 7.7 |
| | | | (94) | (57) | (76) |
| All | 100 mg | 107 | 93 ± 3.6 | 56 ± 17 | 75 ± 7.9 |
| | | | (93) | (NC) | (75) |

Values are presented as “Mean ± SD (Geometric Mean) where N >2;
Projected PD (IDO) inhibition was calculated as Conc/(Conc + EC50) * 100 (%) in which EC = 70 nM;
NC: not calculable due to at least one PK sample was BQL (thus the PD inhibition was projected as 0%);
Time-averaged IDO1 inhibition was calculated using the linear-up-log-down method;
*The number of subjects with calculable Iavg is counted;
DLBCL: diffuse large B-cell lymphoma;
GU: genitourinary cancer;
MEL: melanoma;
NSCLC: non-small-cell lung carcinoma;
OC: ovarian cancer;
RCC: renal cell cancer;
SCCHN: squamous cell carcinoma of the head and neck;
TNBC: triple negative breast cancer.

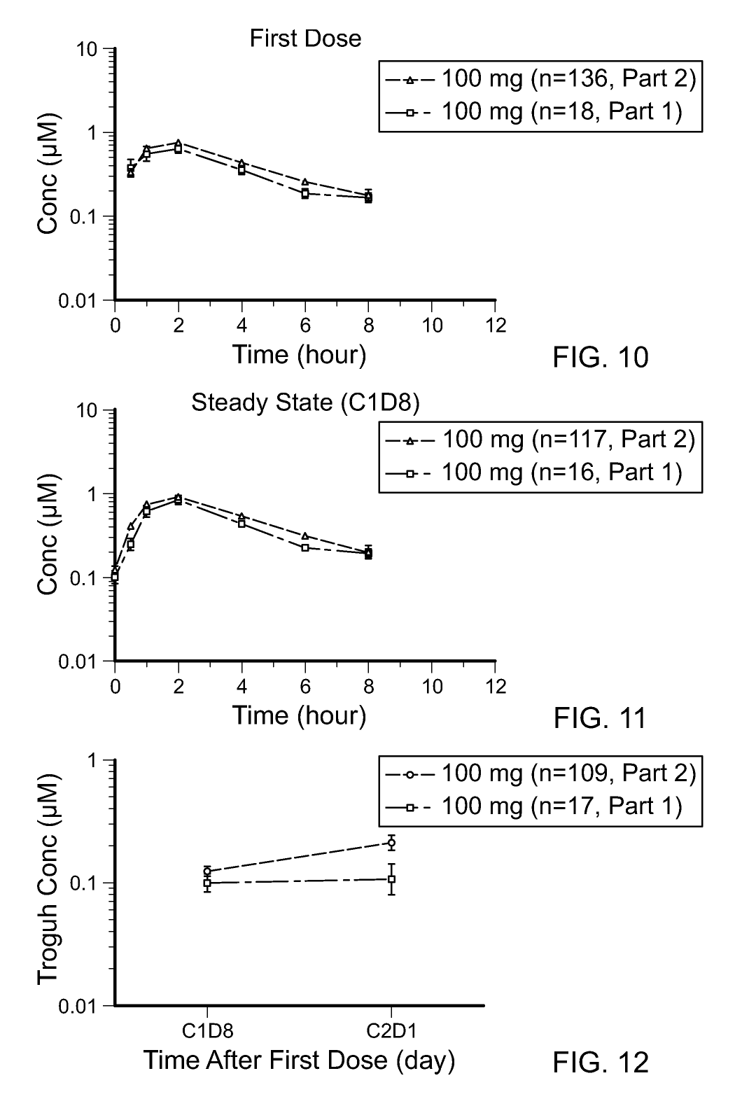

The results are also shown in the figures. FIG. 10 and FIG. 11 show the comparison of Compound 1 plasma concentrations (Mean±SE) following the first dose (FIG. 10) and at steady state (on C1D8, FIG. 11) between part 1 and part 2 in subjects receiving 100 mg BID.

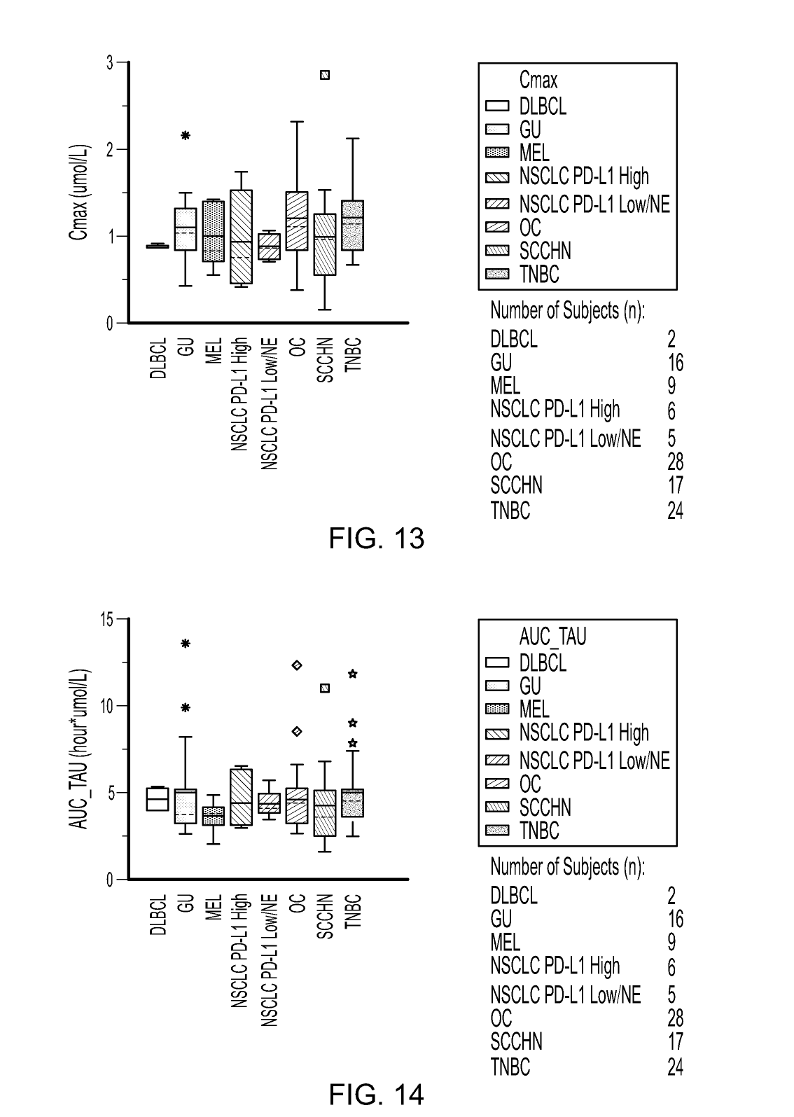

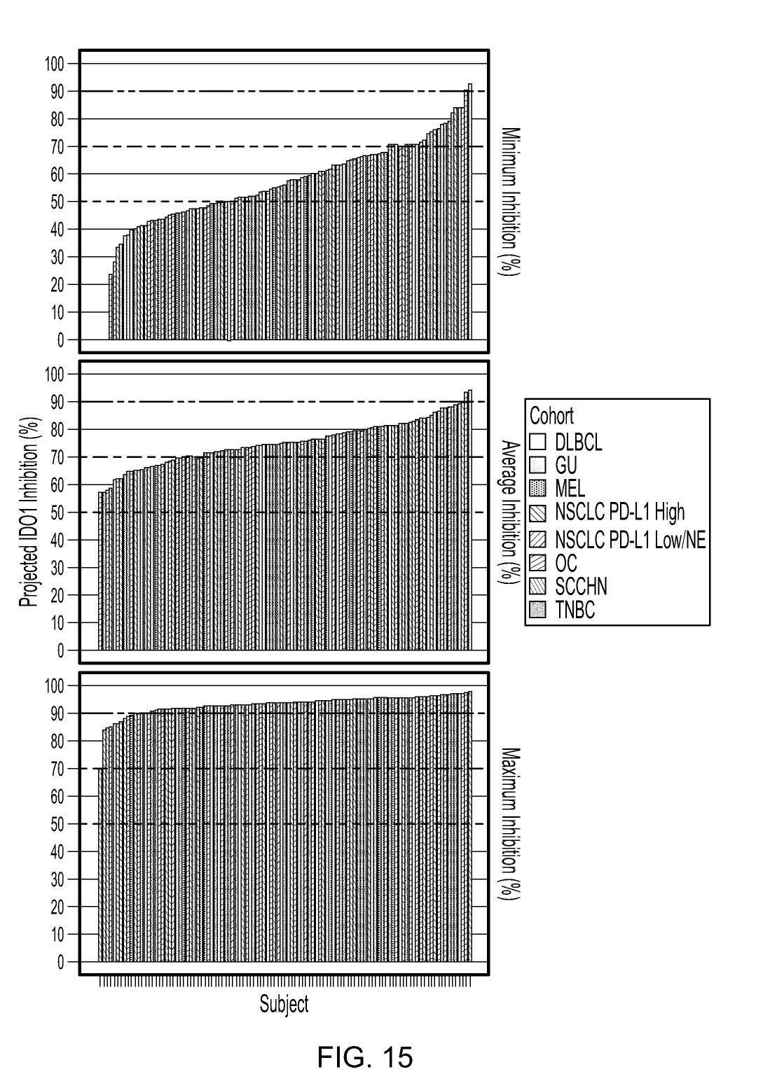

FIG. 12 shows a graph of Compound 1 trough plasma concentrations (Mean±SE) on C1D8 and C2D1 in subjects receiving 100 mg BID. FIG. 13 and FIG. 14 show box plot of Compound 1 at steady state PK for various tumor types. FIG. 15 shows waterfall plots of projected percent IDO1 inhibition at steady state.

Various modifications of the invention, in addition to those described herein, will be apparent to those skilled in the art from the foregoing description. Such modifications are also intended to fall within the scope of the appended claims. Each reference cited in the present disclosure, including all patent, patent applications, and publications, is incorporated herein by reference in its entirety.

What is claimed is:

1. A method of treating cancer in a patient comprising administering to said patient a pharmaceutical composition comprising Compound 1, or a pharmaceutically acceptable salt thereof, 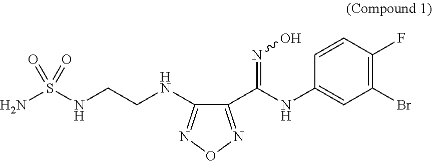 [SMILES: NS(=O)(=O)NCCNc1nonc1C(=NO)Nc1ccc(F)c(Br)c1]
and one or more excipients, in combination with a pharmaceutical composition comprising an inhibitor of an immune checkpoint molecule and one or more excipients, wherein the treating comprises a dosage regimen which attains at steady state, an Iₘᵢₙ of about 50% or greater, or an I_{avg} of about 70% or greater.

2. A method of treating cancer in a patient comprising administering to said patient a pharmaceutical composition comprising Compound 1, or a pharmaceutically acceptable salt thereof, 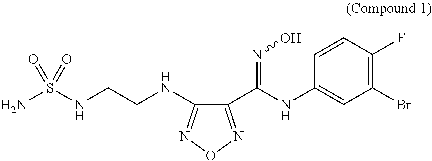 [SMILES: NS(=O)(=O)NCCNc1nonc1C(=NO)Nc1ccc(F)c(Br)c1]
and one or more excipients, in combination with a pharmaceutical composition comprising an inhibitor of an immune checkpoint molecule and one or more excipients, wherein the treating comprises a dosage regimen which attains at steady state:
(3) a Cₘₐₓ from about 0.10 μM to about 10 μM, a Cₘᵢₙ from about 0.01 μM to about 2.0 μM, a Tₘₐₓ of about 1 h to about 6 h and an AUC_{0-τ} from about 1 μM*h to about 50 μM*h; and
(4) an Iₘᵢₙ of about 50% or greater, or an I_{avg} of about 70% or greater.

3. The method of claim 1 or 2, wherein the Iₘᵢₙ is about 50% to about 80%, about 50% to about 70%, or about 50% to about 60%.

4. The method of claim 1 or 2, wherein the Iₘᵢₙ is about 50% to about 60%.

5. The method of claim 1 or 2, wherein the I_{avg} is about 70% to about 90% or about 70% to about 80%.

6. The method of claim 1 or 2, wherein the I_{avg} is about 70% to about 80%.

7. The method of any of claims 1-6, wherein the inhibitor of an immune checkpoint molecule is pembrolizumab.

8. The method of claim 7, wherein the dose regimen comprises from about 25 mg to about 300 mg on a free basis of Compound 1, or a pharmaceutically acceptable salt thereof, administered orally twice daily, and pembrolizumab administered every 21 days.

9. The method of claim 1, wherein the dosage regimen comprises about 100 mg on a free base basis of Compound 1, or a pharmaceutically acceptable salt thereof, which is administered twice daily, which attains, at steady state, an Iₘᵢₙ of about 50% or greater, or an I_{avg} of about 70% or greater.

10. The method of claim 2 wherein the dosage regimen comprises about 100 mg on a free base basis of Compound 1, or a pharmaceutically acceptable salt thereof, which is administered twice daily, which attains, at steady state:
(1) a Cₘₐₓ of about 0.5 μM to about 2.0 μM, Tₘₐₓ of about 2 h and an AUC_{0-τ} of about 4 μM*h to about 7 μM*h; and
(2) an Iₘᵢₙ of about 50% or greater, or an I_{avg} of about 70% or greater.

11. A method of treating cancer in a patient comprising administering to said patient a pharmaceutical composition comprising Compound 1, or a pharmaceutically acceptable salt thereof, 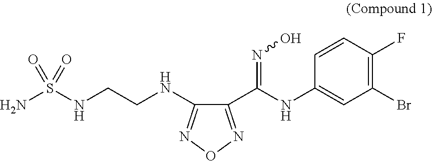 [SMILES: NS(=O)(=O)NCCNc1nonc1C(=NO)Nc1ccc(F)c(Br)c1]
and one or more excipients, wherein the treating comprises a dosage regimen comprising from about 25 mg to about 700 mg on a free basis of Compound 1, or a pharmaceutically acceptable salt thereof, administered orally twice daily, which attains at steady state, a Cₘₐₓ from about 0.10 μM to about 10 μM, a Cₘᵢₙ from about 0.01 μM to about 2.0 μM, a Tₘₐₓ of about 1 h to about 6 h and an AUC_{0-τ} from about 1 μM*h to about 50 μM*h.

12. The method of any one of claims 2 to 8 and 10 to 11, wherein the Cₘₐₓ is about 0.20 μM to about 8.0 μM, about 0.30 μM to about 7.0 μM, about 1.0 μM to about 7.0 μM, about 1.0 μM to about 6.0 μM, about 1.0 μM to about 5.0 μM, about 1.0 μM to about 4.0 μM, or about 1.0 μM to about 3.0 μM.

13. The method of claim 12, wherein the Cₘₐₓ is about 1.0 μM to about 3.0 μM.

14. The method of any one of claims 2 to 8 and 10 to 13, wherein the Tₘₐₓ is about 1 h to about 5 h.

15. The method of claim 14, wherein the Tₘₐₓ is about 2 h to about 3 h.

16. The method of claim 15, wherein the Tₘₐₓ is about 2 h.

17. The method of any one of claims 2 to 8 and 10 to 16, wherein the AUC_{0-τ} is about 1 μM*h to about 40 μM*h, about 1 μM*h to about 36 μM*h, 1 μM*h to about 34 μM*h, about 1 μM*h to about 30 μM*h, about 1 μM*h to about 20 μM*h, about 1 μM*h to about 10 μM*h, about 5 μM*h to about 15 μM*h, or about 5 μM*h to about 10 μM*h.

18. The method of claim 17, wherein the AUC_{0-τ} is about 4 μM*h to about 10 μM*h.

19. The method of claim 18, wherein the AUC_{0-τ} is about 4 μM*h to about 6 μM*h.

20. The method of any one of claims 2 to 8 and 10 to 19, wherein the Cₘᵢₙ is about 0.01 μM to about 2 μM or from about 0.025 μM to about 0.5 μM.

21. A method of treating cancer in a patient comprising administering to said patient a pharmaceutical composition comprising Compound 1, or a pharmaceutically acceptable salt thereof, 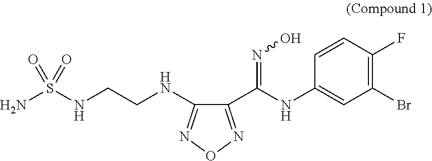 [SMILES: NS(=O)(=O)NCCNc1nonc1C(=NO)Nc1ccc(F)c(Br)c1]
and one or more excipients, wherein the treating comprises a dosage regimen comprising from about 25 mg to about 700 mg on a free basis of Compound 1, or a pharmaceutically acceptable salt thereof, administered orally twice daily.

22. The method of claim 21, wherein said dosage regimen comprises about 50 mg to about 100 mg on a free basis of Compound 1, or a pharmaceutically acceptable salt thereof, administered orally twice daily.

23. The method of claim 21, wherein said dosage regimen comprises about 50 mg on a free basis of Compound 1, or a pharmaceutically acceptable salt thereof, administered orally twice daily.

24. The method of claim 21, wherein said dosage regimen comprises about 100 mg on a free basis of Compound 1, or a pharmaceutically acceptable salt thereof, administered orally twice daily.

25. The method of any one of claims 1 to 21, wherein said dosage regimen comprises about 100 mg to about 700 mg on a free basis of Compound 1, or a pharmaceutically acceptable salt thereof, administered orally twice daily.

26. The method of any one of claims 1 to 21, wherein said dosage regimen comprises about 100 mg to about 400 mg on a free basis of Compound 1, or a pharmaceutically acceptable salt thereof, administered orally twice daily.

27. The method of any one of claims 1 to 21, wherein said dosage regimen comprises about 100 mg to about 300 mg on a free basis of Compound 1, or a pharmaceutically acceptable salt thereof, administered orally twice daily.

28. A method of treating cancer in a patient comprising administering to said patient a pharmaceutical composition comprising Compound 1, 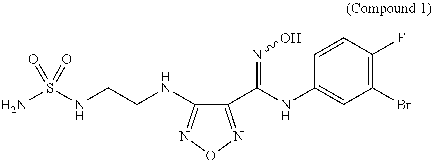 [SMILES: NS(=O)(=O)NCCNc1nonc1C(=NO)Nc1ccc(F)c(Br)c1]
or a pharmaceutically acceptable salt thereof, and one or more excipients, wherein the treating comprises a dosage regimen comprising from about 100 mg to about 300 mg on a free basis of Compound 1, or a pharmaceutically acceptable salt thereof, administered orally twice daily, wherein the dosage regimen attains a trough blood plasma concentration of a fasted individual at steady state that is equal to or greater than IC₅₀ at IDO1 or an average blood plasma concentration of a fasted individual at steady state over the 12 hour interval that is equal to or greater than IC₉₀ at IDO1.

29. The method of any one of claims 1 to 21 and 28, wherein said dosage regimen comprises about 100 mg, about 200 mg, or about 300 mg on a free base basis of Compound 1, or a pharmaceutically acceptable salt thereof, which is administered twice daily.

30. The method of any one of claims 1 to 21 and 28, wherein said dosage regimen comprises about 100 mg on a free base basis of Compound 1, or a pharmaceutically acceptable salt thereof, which is administered twice daily.

31. The method of any one of claims 1 to 21 and 28, wherein dosage regimen comprises about 200 mg on a free base basis of Compound 1, or a pharmaceutically acceptable salt thereof, which is administered twice daily.

32. The method of any one of claims 1 to 21 and 28, wherein said dosage regimen comprises about 300 mg on a free base basis of Compound 1, or a pharmaceutically acceptable salt thereof, which is administered twice daily.

33. The method of any one of claims 1 to 32, wherein each composition is formulated as a tablet.

34. The method of claim 2 or 3, wherein the dosage regimen comprises about 50 mg on a free base basis of Compound 1, or a pharmaceutically acceptable salt thereof, which is administered twice daily, which attains, at steady state, a Cₘₐₓ of about 0.1 μM to about 1.0 μM, a Tₘₐₓ of about 2 h, and an AUC_{0-τ} of about 1 μM*h to about 3 μM*h.

35. The method of claim 2 or 3, wherein said dosage regimen comprises about 100 mg on a free base basis of Compound 1, or a pharmaceutically acceptable salt thereof, which is administered twice-per-day which provides, at steady state, a Cₘₐₓ of about 0.5 μM to about 2.0 μM, Tₘₐₓ of about 2 h and an AUC_{0-τ} of about 4 μM*h to about 7 μM*h.

36. The method of claim 2 or 3, wherein said dosage regimen comprises about 300 mg on a free base basis of Compound 1, or a pharmaceutically acceptable salt thereof, which is administered twice-per-day which provides, at steady state, a Cₘₐₓ of about 1.0 μM to about 3.0 μM, a Tₘₐₓ of about 2 and an AUC_{0-τ} of about 8 μM*h to about 10 μM*h.

37. The method of any one of claims 1 to 36, wherein the patient is in a fasted state.

38. The method of any one of claims 1 to 37, wherein the excipient is selected from lactose monohydrate, microcrystalline cellulose, povidone, croscarmellose sodium, colloidal silicon dioxide, and magnesium stearate.

39. The method of claim 38, wherein lactose monohydrate is present in an amount about 20 wt % to about 35 wt % or about 24 wt % to about 32 wt % of the composition.

40. The method of claim 38 or 39, wherein microcrystalline cellulose is present in an amount about 20 wt % to about 35 wt % or about 22 wt % to about 33 wt % of the composition.

41. The method of any one of claims 38 to 40, wherein povidone is present in an amount about 0.5 wt % to about 1.0 wt % of the composition.

42. The method of claim 41, wherein povidone is present in an amount about 0.8 wt % of the composition.

43. The method of any one of claims 38 to 42, wherein croscarmellose sodium is present in an amount about 1.0 wt % to about 10.0 wt % of the composition.

44. The method of claim 43, wherein croscarmellose sodium is present in an amount about 3 wt % or about 10 wt % of the composition.

45. The method of any one of claims 38 to 44, wherein colloidal silicon dioxide is present in an amount about 0.1 wt % to about 1.0 wt % of the composition.

46. The method of claim 45, wherein colloidal silicon dioxide is present in an amount about 0.6 wt % or about 0.7 wt % of the composition.

47. The method of any one of claims 38 to 46, wherein magnesium stearate is present in an amount about 0.1 wt % to about 1.0 wt % of the composition.

48. The method of claim 47, wherein magnesium stearate is present in an amount about 0.6 wt % of the composition.

49. The method of any one of claims 1 to 48, wherein the cancer is colon cancer, pancreatic cancer, breast cancer, prostate cancer, lung cancer, brain cancer, ovarian cancer, cervical cancer, testicular cancer, renal cancer, head and neck cancer, lymphoma and leukemia.

50. The method of any one of claims 1 to 48, wherein the cancer is solid tumor.

51. The method of any one of claims 1 to 48, wherein the cancer is melanoma, non-small-cell lung carcinoma, transitional cell carcinoma of the genitourinary (GU) tract, renal cell cancer, triple negative breast cancer (TNBC), adenocarcinoma of the endometrium, squamous cell carcinoma of the head and neck (SCCHN), endometrial cancer, gastric cancer, pancreatic ductal adenocarcinoma, diffuse large B-cell lymphoma (DLBCL), or ovarian cancer (OC).

52. The method of any one of claims 1 to 51 further comprising administering one or more inhibitors of an immune checkpoint molecule.

53. The method of claim 52, wherein the inhibitor of an immune checkpoint molecule is an inhibitor of PD-1, PD-L1, PD-L2, CTLA-4, TIM3, LAG3, VISTA, BTLA, TIGIT, LAIR1, CD160, 2B4 and/or TGFR beta.

54. The method of claim 52, wherein the inhibitor of an immune checkpoint molecule is anti-PD1 antibody, anti-PD-L1 antibody, or anti-CTLA-4 antibody.

55. The method of claim 54, wherein the anti-PD1 antibody is nivolumab, pembrolizumab, pidilizumab, SHR-1210, or AMP-224.

56. The method of claim 55, wherein the anti-PD1 antibody is pembrolizumab.

57. The method of claim 56, wherein the pembrolizumab is administered every three weeks.

58. The method of claim 56 or 57, wherein the pembrolizumab is administered at about 2 mg/kg.

59. The method of claim 54, wherein the inhibitor of an immune checkpoint molecule is anti-PD-L1 antibody.

60. The method of claim 59, wherein the anti-PD-L1 antibody is BMS-935559, MED14736, MPDL3280A, or MSB0010718C.

61. The method of claim 54, wherein the inhibitor of an immune checkpoint molecule is anti-CTLA-4 antibody.

62. The method of claim 61, wherein the anti-CTLA-4 antibody is ipilimumab.

63. A method of treating melanoma in a patient comprising administering to said patient a pharmaceutical composition comprising Compound 1, or a pharmaceutically acceptable salt thereof, 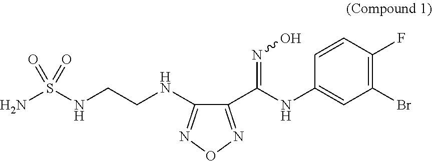 [SMILES: NS(=O)(=O)NCCNc1nonc1C(=NO)Nc1ccc(F)c(Br)c1]
and one or more excipients, in combination with a pharmaceutical composition comprising pembrolizumab and one or more excipients, wherein the treating comprises a dosage regimen comprising from about 25 mg to about 300 mg on a free basis of Compound 1, or a pharmaceutically acceptable salt thereof, administered orally twice daily, and pembrolizumab administered every three weeks.
